(19) United States
(12) Patent Application Publication
(10) Pub. No.: US 2023/0117773 A1
(43) Pub. Date: Apr. 20, 2023

(54) SYSTEMS AND METHODS FOR CONFIGURING AND DEPLOYING A PORTABLE FIELD-DEPLOYABLE BIOSURVEILLANCE KIT

(71) Applicant: NOBLIS, INC., Reston, VA (US)

(72) Inventors: Shane MITCHELL, Reston, VA (US); Sterling Thomas, Woodbridge, VA (US); Masooda Omari, Fairfax, VA (US); Tyler W. Barrus, Reston, VA (US); Mychal Ivancich, Falls Church, VA (US)

(73) Assignee: NOBLIS, INC., Reston, VA (US)

(21) Appl. No.: 17/937,866

(22) Filed: Oct. 4, 2022

Related U.S. Application Data
(60) Provisional application No. 63/256,051, filed on Oct. 15, 2021.

(51) Int. Cl. C12Q 1/6851; C12Q 1/6806; C12Q 1/6811; C12Q 1/6825; C12Q 1/6855; G16B 30/00

(52) U.S. Cl. CPC C12Q 1/6851; C12Q 1/6806; C12Q 1/6811; C12Q 1/6825; C12Q 1/6855; G16B 30/00

(57) ABSTRACT

Portable biosurveillance kits for sequencing and sample identification are provided, and techniques for configuring said kits are provided. A system for configuring a kit may receive data representing a first and second set of nucleic acid sequences, and may generate and store first and second indexes representing the respective sets. The system may then use the indexes to identify conserved-signature sequences that satisfy abundance criteria with respect to the first set and sparsity criteria with respect to the second set. The identified conserved-signature sequences may be stored on (or represented in storage on) a portable sequencing and sample-identification kit, which may compare the conserved-signature sequence to a sample sequence in order to identify the sample sequence.

### CROSS-REFERENCE TO RELATED APPLICATIONS

This application claims the benefit of U.S. Provisional Application No. 63/256,051, filed Oct. 15, 2021, the entire contents of which is incorporated herein by reference.

### REFERENCE TO AN ELECTRONIC SEQUENCE LISTING

The contents of the electronic sequence listing (739642006500seqlsit.xml; Size: 13,403 bytes; and Date of Creation: Sep. 27, 2022) is herein incorporated by reference in its entirety.

### FIELD

This relates generally to systems and methods for configuring and deploying nucleic acid sequencing and sample identification kits, and more specifically to software for use in configuring and using portable and field-deployable DNA sequencing and sample identification kits that may be used without internet connection or line power.

### BACKGROUND

Sequencing and analysis systems for metagenomics samples require various different specialized laboratory devices. The laboratory equipment required for sequencing and analysis of metagenomics samples is typically expensive, bulky, and immobile, and generally requires a line power connection for power and/or an internet connection for performance of data processing and computations. Analysis of metagenomic samples generally requires access to vast libraries of reference data that can only be accessed by providing internet connection and/or large very amounts of local computer storage.

### SUMMARY

As explained above, sequencing and analysis of metagenomic samples is typically performed using immobile laboratory equipment, such as tabletop laboratory devices, which require line power and/or internet connection in order to operate. Accordingly, sequencing and analysis of metagenomics samples according to known methods requires that samples collected from a field environment be packaged and transported from the field environment to a dedicated laboratory for sequencing and analysis. The transportation from the environment to the dedicated laboratory may be time consuming or entirely impracticable, especially if the environment is in a remote or inaccessible location, the transportation process may increase costs of operations, and the transportation process may introduce risks or damage or compromise the collected sample.

An additional challenge in attempting to perform sequencing and analysis of metagenomic samples in the field is that, even if one or more components are field-deployable, they may still depend on internet connections so that they may access vast libraries of reference information that are impractical or impossible to store locally on portable equipment. Thus, metagenomic analysis performed outside a laboratory can be impossible or impractical due to lack of availability of network communications and/or due to slow network communication speeds.

Accordingly, there is a need for systems, methods, and techniques for collection, sequencing, and analysis of metagenomics samples that can be performed in a deployed field environment, without requiring immobile tabletop laboratory equipment and without requiring a network connection to access remotely-stored reference data. Specifically, there is a need for systems for metagenomics processing that can be configured according to different requirements for different use cases, such that the systems can be deployed to perform remote analysis (without network communication) using specially selected and configured reference information that can be efficiently stored and effectively leveraged for the specific needs of the metagenomics use case.

In some embodiments, a portable field-deployable nucleic acid sequencing kit provides lightweight and portable components for sequencing and analysis of metagenomics samples that do not require internet connection or line power, and that may therefore address the above needs. In some embodiments, a kit may be configured to sequence and analyze metagenomics samples without transporting the samples to a dedicated laboratory that includes specialized equipment. The kit may be deployed for use in an environment with limited resources. For example, the kit may be utilized without internet access or line power. The kit may include components for extracting DNA from a metagenomics sample, preparing the extracted DNA for sequencing, quantifying a concentration of the extracted DNA, and direct sequencing of the extracted DNA prepared for sequencing. The components of the kit may be housed within a portable enclosure.

In some embodiments, the portable kit may be configured to decrease turnaround time between sample collection and analysis. The decreased turnaround time supports real-time surveillance of samples collected from an inaccessible or sensitive field environment.

In some embodiments, the portable kit may be selectively configurable in accordance with one or more specific use cases, allowing users to configure the kit for deployment for a certain purpose and/or in a certain region. For example, the kit may be specifically configured for attempted identification of a specific organism, set of organisms, and/or type of organisms. Furthermore, the kit may be specifically configured for attempted identification of the target organism(s) while distinguishing one or more other organisms and/or types of organisms that are known or expected to be present in an environment (and in metagenomic samples) alongside the target organism(s).

In some embodiments, the portable kit may be configured for deployment and/or use by selecting a set of one or more conserved-signature sequences to be stored on (or represented by one or more data structures stored on) the portable kit. The conserved signature sequences may, in some embodiments, be identified by use of a set of indexes, where a first index represents one or more target organisms that are sought to be identified in a use case, and where a second index represents one or more organisms that are sought to be distinguished in a use case. The system may use the first index to identify one or more conserved regions that appear in every nucleic acid sequence in a set representing the targeted organism(s). The system may then use the second index to confirm whether the identified conserved sequence does or does not appear in any of the nucleic acid sequences in a set representing the organism(s) to be distinguished. If it is confirmed that the conserved sequence does not appear in the set representing the organism(s) to be distinguished, then the sequence may be identified as a conserved-signature sequence and may be selected for deployment in the contemplated use case. Selected conserved-signature sequences may then be transmitted to the portable kit for storage thereon, optionally along with associated metadata. Alternatively or additionally, one or more data structures representing said selected conserved-signature sequences, such as a probabilistic data structure representing one or more conserved signature sequences, may be transmitted to the portable kit for storage thereon.

In some embodiments, the system may be configured such that a user may selectively configure a portable kit by identifying a set of conserved-signature sequences based on one or more user inputs. In some embodiments, a first user input, for example executed at a computing system for configuring the kit, may indicate an identity of one or more organisms to be targeted. In some embodiments, a user input may indicate one or more target organisms by a user uploading one or more complete or partial nucleic acid sequences for said target organism. In some embodiments, a user input may indicate one or more target organisms by a user selecting the one or more target organisms from a list or menu. In some embodiments, a user input may indicate one or more target organisms by a user selecting one or more classifications (e.g., organism groups, organism types) from a list or menu. In some embodiments, a user input may indicate one or more target organisms by a user selecting one or more characteristics or traits and the system selecting target organisms that match the indicated characteristics or traits. In some embodiments, the system may build one or more indexes (e.g., the “first index” referenced above) in accordance with the first user input executed by the user indicating the one or more target organisms. Similarly, a second user input may indicate an identity of one or more organisms to be distinguished, and the system may build one or more indexes (e.g., the “second index” referenced above) in accordance with the second user input executed by the user indicating the one or more organisms to be distinguished.

After the identified conserved-signature sequences have been stored locally on the portable kit, the kit may then be used for sequence identification without network connection to a large database. For example, a locally collected sample may be used to generate a plurality of sequences (e.g., k-mers) representing a nucleic acid sequence associated with the sample. The sample nucleic acid sequences may then be compared to the conserved-signature sequences stored locally on the portable kit in order to determine whether there is a match. In the event a match is identified, one or more coverage metrics may be generated, for example indicating how many and/or what percentage of the sample sequences (e.g., sample k-mers) match a conserved-signature sequence for one or more organisms.

Optionally, identified conserved-signature sequences may be represented in storage on the portable kit using one or more probabilistic data structures, such as Bloom filters. A probabilistic data structure stored on the portable kit may confer the advantage that it may help to obfuscate the identity of one or more sequences represented by the data structure, which may be desirable in situations in which the conserved-signature sequences are proprietary and/or in which the conserved-signature sequences present security concerns. In situations in which the portable kit stores one or more probabilistic data structures, the sample sequences may be used to query a probabilistic data structure to responsively generate an indication as to whether the sample sequence is (a) probably included in the set of sequences represented by the probabilistic data structure or (b) definitely not included in the set of sequences represented by the probabilistic data structure. Additional processing of probable matches may thereafter be performed locally or remotely, or the probable match itself may be used to conclude that a match has occurred and/or to generate one or more coverage metrics.

In some embodiments, a portable kit for nucleic acid sequencing and sample identification is provided, the portable kit comprising: a DNA extraction system configured to perform a DNA extraction protocol to extract DNA from a sample; a DNA sequencing preparation system configured to perform a DNA sequencing preparation protocol on the extracted DNA; a sequencer system configured to generate sample nucleic acid sequence data for the extracted DNA; memory storing reference nucleic acid data representing a plurality of reference nucleic acid sequences; one or more processors configured to compare the sample nucleic acid sequence data to the reference nucleic acid data to generate output data indicating one or more organisms with which the sample is determined to correspond; and a portable enclosure configured to house the DNA extraction system, the sequencer preparation system, the sequencer system, the memory, and the one or more processors.

In some embodiments of the portable kit, the reference nucleic acid data represents one or more target regions identified by: determining, by a first index comprising data representing a first set of nucleic acid sequences, that the target region is a conserved region appearing in every nucleic acid sequence in the first set; and confirming, by the second index comprising data representing a second set of nucleic acid sequences, that the conserved region appears in none of the nucleic acid sequences in the second set.

In some embodiments of the portable kit, the reference nucleic acid data comprises sequence data and associated metadata, wherein the associated metadata indicates an organism associated with the target region.

In some embodiments of the portable kit, the associated metadata indicates a type of organism associated with the target region.

In some embodiments of the portable kit, the reference nucleic acid data comprises a probabilistic data structure that represents one or more of the plurality of reference nucleic acid sequences as members of a set.

In some embodiments of the portable kit, comparing the sample nucleic acid sequence data to the reference nucleic acid data comprises querying the probabilistic data structure by data representing the sample nucleic acid sequence to responsively generate data indicating whether the sample nucleic acid sequence is a member of the set.

In some embodiments of the portable kit, the probabilistic data structure is stored as part of a multi-level data structure comprising a plurality of hierarchically-interrelated probabilistic data structures; probabilistic data structures in a first level of the multi-level data structure represent respective sets of the plurality of reference nucleic acid sequences; and probabilistic data structures in a second level of the multi-level data structure represent respective subsets of the sets of the plurality of reference nucleic acid sequences.

In some embodiments of the portable kit: data structures in the first level of the multi-level data structure represent respective sets of the plurality of reference nucleic acid sequences that are associated with a respective type of organism; and data structures in the second level of the multi-level data structure represent respective sets of the plurality of reference nucleic acid sequences that are associated with a respective organism.

In some embodiments of the portable kit, the output data comprises ranking data indicating a respective match strength for each of the one or more organisms with which the sample is determined to correspond.

In some embodiments of the portable kit, the respective match strength is determined based on a number of sequences in the reference nucleic acid data to which the sample nucleic acid sequence data is determined to correspond.

In some embodiments of the portable kit, the memory is configured to be selectively loaded with different sets of reference nucleic acid data representing different pluralities of reference nucleic acid sequences.

In some embodiments of the portable kit, one of the different pluralities of reference nucleic acid sequences corresponds to a predefined type of organism, including one or more of the following: biological warfare agents, food pathogens, viruses, bacteria, fungi, mammalian, and harmful agents.

In some embodiments of the portable kit, the portable kit comprises a first centrifuge and a portable power supply, wherein: the first centrifuge is configured to ramp to a target speed at a first ramp rate when drawing power from the portable power supply, and the first centrifuge is configured to ramp to the target speed at a second ramp rate, faster than the first ramp rate, when drawing power from a source of line power.

In some embodiments of the portable kit, ramping at the first ramp rate comprises increasing from an initial speed to the target speed in predetermined increments.

In some embodiments of the portable kit, the sequencer system comprises a sequencer device, a heating device positioned external to the sequencing device, and an insulated casing that houses the sequencing device and the heating device.

In some embodiments, a first system for configuring a kit for nucleic acid sequencing and sample identification is provided, the first system comprising: a first set of one or more processors configured to: receive genomic data representing a first set of one or more nucleic acid sequences; create and store data in a first index representing a first set of nucleic acid sequences; receive genomic data representing a second set of one or more nucleic acid sequences; create and store data in a second index representing the second set of nucleic acid sequences; and identify a target region to serve as a nucleic acid reference sequence that corresponds to one or more of the nucleic acid sequences in the first set and that discriminates against one or more of the nucleic acid sequences in the second set, wherein the identifying comprises: identifying, by the first index, the target region as a conserved region appearing in every nucleic acid sequence in the first set; and confirming, by the second index, that the conserved region appears in none of the nucleic acid sequences in the second set; and a portable kit for nucleic acid sequencing and sample identification, the portable kit comprising a second set of one or more processors and memory; wherein the first set of one or more processors are configured to cause transmission of data representing the target region to the portable kit for storage on the memory; wherein the second set of one or more processors are configured to compare the target region to a sample nucleic acid sequence to determine whether the target region matches the sample nucleic acid sequence.

In some embodiments of the first system: receiving the genomic data representing the first set comprises receiving a first user input indicating the one or more nucleic acid sequences; and receiving the genomic data representing the first set comprises receiving a second user input indicating the one or more nucleic acid sequences.

In some embodiments of the first system, the first user input comprises selection of an organism from a menu.

In some embodiments of the first system, the first user input comprises selection of a type of organisms from a menu.

In some embodiments of the first system, the second user input comprises selection of an organism from a menu.

In some embodiments of the first system, the second user input comprises selection of a type of organisms from a menu.

In some embodiments of the first system, the first set of one or more processors configured to receive a length input indicating a base length for the target region to be identified.

In some embodiments of the first system: the first set of one or more processors is configured to receive an input indicating an index base-length to be used in creation of the first index; and creating and storing data in a first index representing the first set of nucleic acid sequences comprises representing the first set of nucleic acid sequences using subsequences having a length equal to the indicated index base-length.

In some embodiments of the first system, the input indicating the index base-length comprises one or more of the following: a user input explicitly specifying a number of bases; data characterizing processing resources of the first set of one or more processors; and data characterizing storage resources available for storage of the first index.

In some embodiments of the first system: the first set of one or more processors is configured to receive an input indicating a target region base-length criteria to be used in identification of the target region; and identifying the target region comprises ensuring that the identified target region has a length that complies with the indicated target region base-length criteria.

In some embodiments of the first system, the input indicating the target region base-length criteria comprises one or more of the following: a user input explicitly specifying a number of bases; data characterizing processing resources of the first set of one or more processors; data characterizing storage resources available for storage of the first index; data characterizing processing resources of the second set of one or more processors; and data characterizing storage resources available on the memory for storage of the conserved-signature sequences on the portable kit.

In some embodiments of the first system, the input indicating the target region base-length criteria comprises data characterizing a base length of sample nucleic acid sequences generated by a sequencing system of the portable kit.

In some embodiments of the first system, the data representing the target region comprises sequence data and associated metadata, wherein the associated metadata indicates an organism associated with the target region.

In some embodiments of the first system, the data representing the target region comprises sequence data and associated metadata, wherein the associated metadata indicates a type of organism associated with the target region.

In some embodiments of the first system, the data representing the target region comprises a probabilistic data structure that represents the target region as a member of a set.

In some embodiments of the first system, comparing the target region to a sample nucleic acid sequence comprises querying the probabilistic data structure by data representing the sample nucleic acid sequence to responsively generate data indicating whether the sample nucleic acid sequence is a member of the set.

In some embodiments of the first system, the first set of one or more processors are configured to: receive a false-positivity probability input; and of the probabilistic data structure; and set a false-positivity rate for the probabilistic data structure in accordance with the false-positivity probability input.

In some embodiments of the first system: the probabilistic data structure is stored as part of a multi-level data structure comprising a plurality of hierarchically-interrelated probabilistic data structures; probabilistic data structures in a first level of the multi-level data structure represent respective sets of the plurality of reference nucleic acid sequences; and probabilistic data structures in a second level of the multi-level data structure represent respective subsets of the sets of the plurality of reference nucleic acid sequences.

In some embodiments of the first system: data structures in the first level of the multi-level data structure represent respective sets of the plurality of reference nucleic acid sequences that are associated with a respective type of organism; and data structures in the second level of the multi-level data structure represent respective sets of the plurality of reference nucleic acid sequences that are associated with a respective organism.

In some embodiments of the first system, the first set of one or more processors are configured to: receive a multi-level data-structure arrangement input; and define an arrangement of the multi-level data structure in accordance with the multi-level data-structure arrangement input.

In some embodiments of the first system: the data representing the target region comprises an index representing the target; the index comprises a plurality of data structures representing respective sub-string of the target region; and the respective data structures are stored in the index and indicate an identity of the target region, a permutation of bases forming the sub-string of the target region, and a position of the sub-string in the target region.

In some embodiments of the first system, comparing the target region to a sample nucleic acid sequence comprises determining whether the index stores a data structure associated with a sub-string of the sample nucleic acid sequence.

In some embodiments of the first system, creating and storing data in the first index comprises: for each of the nucleic acid sequences in the first set, dividing the nucleic acid sequence into a plurality of sub-strings; for each of the plurality of sub-strings, storing a data structure in the first index, wherein: the data structure indicates an identity of the nucleic acid sequence, a permutation of bases forming the sub-string, and a position of the sub-string in the nucleic acid sequence.

In some embodiments, a non-transitory computer-readable storage medium storing instructions for configuring a kit for nucleic acid sequencing and sample identification is provided, wherein the instructions are configured to be executed by a system comprising one or more processors to cause the system to: receive genomic data representing a first set of one or more nucleic acid sequences; create and store data in a first index representing a first set of nucleic acid sequences; receive genomic data representing a second set of one or more nucleic acid sequences; create and store data in a second index representing the second set of nucleic acid sequences; identify a target region to serve as a nucleic acid reference sequence that corresponds to one or more of the nucleic acid sequences in the first set and that discriminates against one or more of the nucleic acid sequences in the second set, wherein the identifying comprises: identifying, by the first index, the target region as a conserved region appearing in every nucleic acid sequence in the first set; and confirming, by the second index, that the conserved region appears in none of the nucleic acid sequences in the second set; and transmit data representing the target region to a portable kit for storage on memory of the portable kit, wherein the portable kit is configured to compare the target region to a sample nucleic acid sequence to determine whether the target region matches the sample nucleic acid sequence.

In some embodiments, a method for configuring a kit for nucleic acid sequencing and sample identification is provided, the method performed at a system comprising one or more processors, the method comprising: receiving genomic data representing a first set of one or more nucleic acid sequences; creating and storing data in a first index representing a first set of nucleic acid sequences; receiving genomic data representing a second set of one or more nucleic acid sequences; creating and storing data in a second index representing the second set of nucleic acid sequences; identifying a target region to serve as a nucleic acid reference sequence that corresponds to one or more of the nucleic acid sequences in the first set and that discriminates against one or more of the nucleic acid sequences in the second set, wherein the identifying comprises: identifying, by the first index, the target region as a conserved region appearing in every nucleic acid sequence in the first set; and confirming, by the second index, that the conserved region appears in none of the nucleic acid sequences in the second set; and transmitting data representing the target region to a portable kit for storage on memory of the portable kit, wherein the portable kit is configured to compare the target region to a sample nucleic acid sequence to determine whether the target region matches the sample nucleic acid sequence.

In some embodiments, a second system for configuring a kit for nucleic acid sequencing and sample identification is provided, the second system comprising one or more processors configured to: receive genomic data representing a first set of one or more nucleic acid sequences; create and store data in a first index representing a first set of nucleic acid sequences; receive genomic data representing a second set of one or more nucleic acid sequences; create and store data in a second index representing the second set of nucleic acid sequences; identify a target region to serve as a nucleic acid reference sequence that corresponds to one or more of the nucleic acid sequences in the first set and that discriminates against one or more of the nucleic acid sequences in the second set, wherein the identifying comprises: identifying, by the first index, the target region as a conserved region appearing in every nucleic acid sequence in the first set; and confirming, by the second index, that the conserved region appears in none of the nucleic acid sequences in the second set; and transmit data representing the target region to a portable kit for storage on memory of the portable kit, wherein the portable kit is configured to compare the target region to a sample nucleic acid sequence to determine whether the target region matches the sample nucleic acid sequence.

In some embodiments, any one or more of the systems, methods, kits, computer-readable storage media, and/or devices described above may be combined, in whole or in part, with all or part of one another and/or with all or part of any other one or more of the systems, methods, kits, computer readable storage media, and/or devices disclosed herein.

### BRIEF DESCRIPTION OF THE FIGURES

The invention will now be described, by way of example only, with reference to the accompanying drawings, in which:

FIG. 1 shows a diagrammatic representation of a portable nucleic acid sequencing and sample identification kit and a system for configuration of the kit, in accordance with some embodiments.

FIG. 2 shows a representation of an index of reference permutations of nucleic acid sequence portions (SEQ ID NOS: 5, 7, 3, 8, 4, 5, 10, 11, 13, 14, and 6, respectively, in order of appearance), in accordance with some embodiments.

FIG. 3 shows a method for configuring a portable nucleic acid sequencing and sample identification kit, in accordance with some embodiments.

FIG. 4 shows a method for performing nucleic acid sequencing using a portable nucleic acid sequencing kit, in accordance with some embodiments.

FIG. 5 shows a computer, in accordance with some embodiments.

### DETAILED DESCRIPTION

The following description sets forth exemplary systems, methods, techniques, parameters, and the like. It should be recognized, however, that such description is not intended as a limitation on the scope of the present disclosure but is instead provided as a description of exemplary embodiments. In the following description, reference is made to the accompanying drawings in which are shown, by way of illustration, specific embodiments that can be practiced. It is to be understood that other embodiments and examples can be practiced, and changes can be made, without departing from the scope of the disclosure.

In addition, it is also to be understood that the singular forms “a”, “an,” and “the” used in the following description are intended to include the plural forms as well, unless the context clearly indicates otherwise. It is also to be understood that the term “and/or,” as used herein, refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units, but do not preclude the presence or addition of one or more other features, integers, steps, operations, elements, components, units, and/or groups thereof.

In some embodiments, a portable kit for DNA sequencing and sample identification is provided, where the kit includes one or more components for performing DNA extraction, DNA quantification, and DNA sequencing of a sample collected in the field. In addition, the kit includes a portable computing device and a portable computer storage medium that are configured to enable the kit to perform efficient and effective sample identification of sequenced nucleic acid sequences generated by the sequencing system.

As described below, the portable computing device and portable computer storage medium may be selectively configurable based on one or more user inputs to be able to perform efficient and effective identification of a predefined organism or set of organisms, including by being configured to efficiently and effectively distinguish one or more other organisms that may be expected to be present in an environment in which the kit is deployed. Configuring the kit for deployment may include identifying one or more conserved-signature sequences for storage on (or representation on) the portable computer storage medium.

Conserved signature sequences may be identified, in some embodiments, by a kit configuration engine communicatively coupled to one or more reference nucleic acid databases. The kit configuration engine may select, based on user input, a first set of nucleic acid sequences associated with organisms to be identified and a second set of nucleic acid sequences associated with organisms to be distinguished. The kit configuration engine may construct an index based on the first set and an index based on the second set. The kit configuration engine may then use the two indexes to identify conserved-signature sequences, which may be sequences that appear in every one (or a percentage above a threshold amount) of the sequences represented in the first index and that do not appear in any (or appear in a percentage below a threshold amount) of the sequences represented in the second index. The conserved-signature sequences, or representations thereof, may then be transmitted to and stored on the portable computer storage medium of the kit, such that the kit can make comparisons against the locally-stored conserved-signature sequences when the kit is deployed.

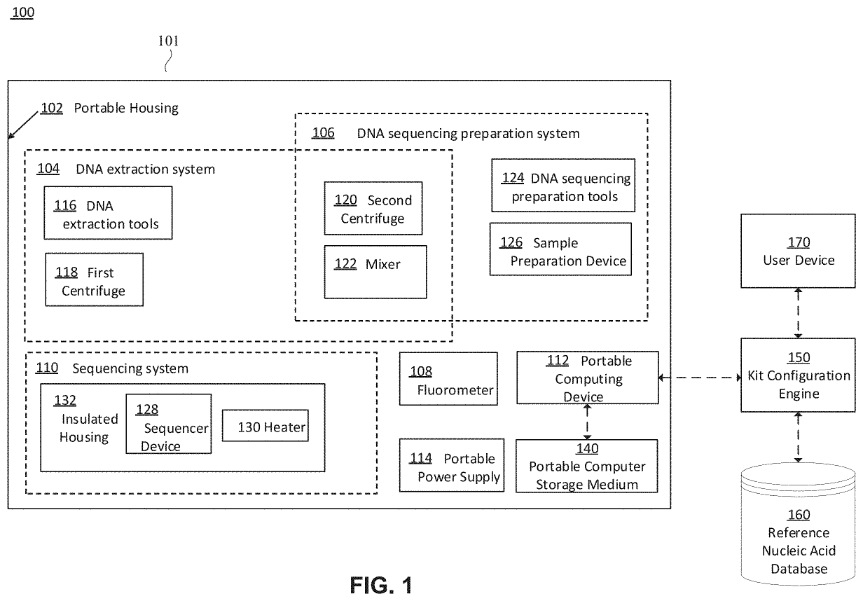

FIG. 1 shows a diagrammatic representation of a portable nucleic acid sequencing and sample identification kit 101 and a system 100 for configuration of the kit, in accordance with some embodiments. As shown in FIG. 1, system 100 may include kit 101, kit configuration engine 150, reference nucleic acid database 160, and user device 170.

In some embodiments, kit 101 includes components configured to extract DNA, quantify DNA, and sequence DNA of metagenomics samples. As shown, the components may include DNA extraction system 104, DNA sequencing preparation system 106, and sequencing system 110, each of which may include one or more sub-components. The portable field sequencing kit may include a casing for housing, transporting, and protecting the various components of the kit as they are transported and deployed in the field.

As shown in FIG. 1, kit 101 may additionally include portable power supply 114, portable computing device 112, and portable computer storage medium 140. Portable computing device may comprise one or more processors that are capable of being operated without line power, for example by drawing power from portable power supply 114. Portable computing device 112 may be configured to control operation of any one or more of the components included in kit 101, including by causing the one or more components to execute any one or more of the protocols (e.g., extraction protocols, sequencing preparation protocols, and/or sequencing protocols) described herein.

Portable computing device 112 may be configured to perform one or more sample identification protocols in which a nucleic acid sequence extracted from a collected sample—e.g., a sequence generated by sequencing system 110—is identified by being determined to be associated with one or more organisms, groups of organisms, or characteristics. In some embodiments, identification of a sample nucleic acid sequence may include comparison of the sample nucleic acid sequence to determine that the sample sequence matches one or more reference nucleic acid sequences, which may be stored (or otherwise represented on) portable computer storage medium 140. Determination of whether a sample nucleic acid sequence corresponds to a reference nucleic acid sequence stored on storage medium 140 may comprise determining whether the sequences are an exact match, a near match (e.g., above a threshold percentage of matching bases), and/or a probable match (e.g., a determination that the sample sequence is probably represented by a probabilistic data structure).

As shown in FIG. 1, portable computing device 112 may be communicatively coupled (e.g., intermittently communicatively coupled, e.g., by wired and/or wireless network communication) with kit configuration engine 150. Kit configuration engine 150 may comprises one or more processors and may be communicatively coupled with reference nucleic acid database 160 and user device 170. As described below in further detail, kit configuration engine may be configured to determine and/or select one or more reference nucleic acid sequences (which may include subsequences of other sequences) for deployment on kit 101 (e.g., for storage on medium 140).

Engine 150 may receive one or more user inputs (e.g., executed by a user of device 170), wherein said user inputs may specify one or more nucleic acid sequences from amongst a plurality of nucleic acid sequence stored on reference nucleic acid database 160. For example, a user of user device 170 may enter one or more inputs via a graphical user interface displayed by user device 170. The user input(s) may comprise entry of text into a field and/or selection of an item from a drop-down or other menu. The user input(s) may indicate one or more nucleic acid sequences. The user input(s) may indicate an identity (e.g., a name) of one or more organisms. The user input(s) may indicate an identity (e.g., a name) or one or more groups of organisms, such as an organism type. Organism types may include, for example, biological warfare agents, food pathogens, viruses, bacteria, fungi, mammalian, and/or harmful agents. The user input(s) may indicate a trait (e.g., a phenotype) of one or more organisms. The user input(s) may indicate a trait of one or more nucleic acid sequences. Based on the user input(s), engine 150 may select a subset of the nucleic acid sequences stored on database 160, for example by using the user inputs to filter the nucleic acid sequences stored on database 160 to select those that satisfy one or more criteria specified by the user input(s).

(Alternatively or additionally to selecting nucleic acid sequences from database 160 based on input(s) by a user of device 170, a user or system may upload one or more nucleic acid sequences directly to engine 150, and the uploaded sequences may be used for index generation and conserved-signature sequence identification as described herein.)

In some embodiments, engine 150 may select, based on the user input(s) received, two sets of nucleic acid sequences. A first set of nucleic acid sequences may be selected based on a first set of one or more user inputs specifying nucleic acid sequences to be searched for, and a second set of nucleic acid sequences may be selected based on a second set of one or more user inputs specifying nucleic acid sequences to be distinguished. For example, the first set of user inputs may specify one or more organisms that a user wishes to search for and identify, while the second set of user inputs may specify one or more organisms that the user wishes to distinguish. A user may use the second set of user inputs to specify one or more organisms that are expected to be included in samples collected in the field but that are not a target organism.

In order to configure the kit in accordance with the user inputs to target the desired target organisms (to return positive matches for those organisms) and to distinguish the specified set of organisms (to not return false positive matches for those organisms), engine 150 may analyze the selected first set and second set of nucleic acid sequences in order to identify one or more conserved-signature sequences. A conserved-signature sequence may be a sequence (e.g., a sequence of a specified length, which may also optionally be specified by user input) that is meets abundance criteria in the first set and that meets sparsity criteria in the second set. For example, the system may apply abundance criteria to determine that a sequence (e.g., a string of length k) is present in every one of the nucleic acid sequences in the first set (or that it is present in a predefined minimum percentage of sequences in the first set) and is therefore a conserved sequence. Similarly, the system may apply sparsity criteria to determine that a sequence (e.g., a string of length k, and/or an identified conserved sequence) is not present in any of the nucleic acid sequences in the second set (or that it is only present in a predefined maximum percentage of sequences in the second set) and is therefore a signature sequence.

In some embodiments, the system may assess abundance criteria and/or sparsity criteria by building one or more indexes to represent the sets of nucleic acid sequences, and then analyzing the content of those indexes. For example, a first index representing the first set of nucleic acid sequences may be constructed, and a second index representing the second set of nucleic acid sequences may be constructed. In some embodiments, kit configuration engine 150 may generate and/or configure one or more indexes and may then analyze the content of said one or more indexes to select conserved-signature sequences.

In some embodiments, engine 150 may create, receive, store, and/or provide an index of a nucleic acid sequence or an amino acid sequence. The index may include a plurality of elements, with each element corresponding to a permutation of a nucleic acid sequence or an amino acid sequence (or another type of sequence). Engine 150 may implement the index using a variety of data structures, such as databases, matrices, arrays, linked lists, trees, and the like. The choice of data structures may vary and is not critical to any embodiment. Engine 150 may store the index in any suitable electronic storage medium, including but not limited to database 160. More specifically, the index may be stored on hard disk; engine 150 may also load the index into RAM for increased performance.

An example nucleic acid sequence is shown in Table 1, below.

TABLE 1
Example Nucleic Acid Sequence

1234568790123456879012345687901234568790
ATTGCTTCCATGGGTC (SEQ ID NO: 1)

As shown in Table 1, a nucleic acid sequence contains various combinations of the bases adenine, guanine, thymine, and cytosine, represented by the letters “A,” “G,” “T,” and “C,” respectively. The numerical digits included in Table 1 enable convenient identification of the positions of the different bases appearing in the sequence. For example, the base adenine appears in positions 1 and 10 of the sequence appearing in Table 1, which is 16 bases in length.

An example amino acid sequence is shown in Table 2, below.

TABLE 2
Example Amino Acid Sequence

1234568790123456879012345687901234568790
DVQMIQSPSSLSASLGDIVTMTCQASQGTSINLNW
FQQKPGKAPKLLIYGSSNLEDGVPSRFSGSRYGTD
FTLTISSLEDEDLATYFCLQHSYLPYTFGGGTKLEI
KR (SEQ ID NO: 2)

As shown in Table 2, an amino acid sequence may contain various combinations of the bases, as represented by the one-letter abbreviations for the standard amino acids. The amino acid sequence shown in Table 2 recites amino acids selected from the 22 standard (proteinogenic or natural) amino acids, but sequences comprising nonstandard amino acid sequences may also be used.

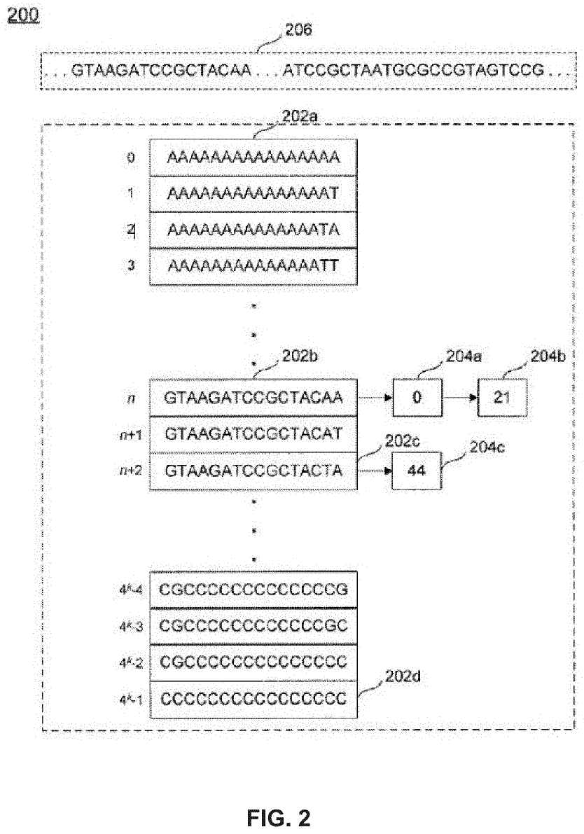

FIG. 2 illustrates an index 200 of a nucleic acid sequence, consistent with some embodiments disclosed herein. Although FIG. 2 illustrates use of nucleic acid sequences, one of ordinary skill in the art would understand how such an example would apply to other types of sequences, such as RNA sequences (e.g., involving the bases adenine, guanine, uracil, and cytosine), sequences of artificially synthesized polymers (such as PNA), and amino acid sequences, including standard (proteinogeneic or natural) and non-standard (non-proteinogenic or non-natural) amino acids.

As shown in FIG. 2, index 200 includes a plurality of elements corresponding to various permutations of nucleic acid sequences. In the case of FIG. 2, each permutation is 16 bases in length, resulting in an index with 4¹⁶ or 4,294,967,296 elements (note that each base of a nucleic acid sequence is one of four types). More generally, the size or the number of elements of index 200 is equal to 4^{k}, where k is the length, in bases, of each permutation.

As shown to the left of each element in FIG. 2, a given element of the index may be referred to by its position number. For example, as illustrated in FIG. 2, position “0” refers to the element corresponding to the permutation “AAAAAAAAAAAAAAAA (SEQ ID NO: 3)” (which is also indicated by reference number 202a), position “3” refers to the element corresponding to the permutation “AAAAAAAAAAAAAATT (SEQ ID NO: 4),” (and position “n” refers to the element corresponding to the permutation “GTAAGATCCGCTACAA (SEQ ID NO: 5),” (which is also indicated by reference number 202b). Because the index may have up to 4^{k} elements, as described above, the elements may be referenced beginning from position “0” to position “4^{k-1}.”

In some embodiments, index 200 may contain a number of elements fewer than the number of possible permutations of sequences of a predetermined length. For instance, computer 101 may use statistical and/or probabilistic methods to reduce the number of elements so that only certain nucleic acid sequences (e.g., those most likely to occur) are included in the index. Such an index has the potential advantage of increased computational efficiency and reduction in memory requirements.

Continuing on, reference numbers 202a, 202b, 202c, and 202d of FIG. 2 represent different elements (e.g., elements “0,” “n,” “n+2,” and “4^{k-1},” respectively) appearing in index 200. In some embodiments, reference numbers 204a, 204b, and 204c describe additional features of index 200. In particular, these reference numbers indicate position data corresponding to certain elements of the index, e.g., reference numbers 204a and 204b indicate position data stored in element 202b, and reference number 204c indicates position data stored in element 202c. In some embodiments, such as those in which the index includes reference numbers 204 or other position data, the index may provide information about one or more specific nucleic acid sequences; thus, the position data stored in an element may reflect a position or location of the nucleic acid sequence in which the corresponding permutation occurs. For instance, as shown in FIG. 2, reference numbers 204a and 204b indicate that the permutation corresponding to element n of the index, “GTAAGATCCGCTACAA (SEQ ID NO: 5),” appears beginning at positions “0” and “21” of the nucleic acid sequence 206. Similarly, reference number 204c indicates that the permutation corresponding to element n+2 of the index, “GTAAGATCCGCTACTA (SEQ ID NO: 9),” appears beginning at position “44” of the nucleic acid sequence 206.

In some embodiments, as discussed further below, reference numbers for distinct nucleic acid sequences may be loaded into the same index, such that the index may reflect position data for sub-strings of multiple nucleic acid sequences. In some such embodiments, each reference number include both position data indicating the position of the permutation within the nucleic acid sequence as well as metadata identifying the nucleic acid sequence to which the position data corresponds.

The nucleic acid elemental sequences may be received from an underlying nucleic acid sample sequence, which may be much greater in length (e.g., millions or billions of bases).

In some embodiments, an index may not contain any location information such as reference numbers 204 and may not contain other information that is specifically related to a particular nucleic acid sequence. That is, in some embodiments, an index may be a generalized index that represents only the elements of the index and corresponding reference numbers 202, such as elements “AAAAAAAAAAAAAAAA (SEQ ID NO: 3)” through “CCCCCCCCCCCCCCCC (SEQ ID NO: 6)” and the corresponding reference numbers 0 through 4^{k-1}. In some embodiments, such an index may be a blank slate for decoding position data and/or to which position data and/or reference numbers may thereafter be saved (the process of inserting position data corresponding to a nucleic acid sequence into an index may be called “seeding” the index.).

In some embodiments, an index contain an exhaustive listing of every mathematically possible permutation of bases for one or more given element-lengths k, representing every mathematically possible element of the given length(s) and corresponding reference numbers. In some embodiments, an index may contain less than every mathematically possible permutation; for example, an index may contain every practically possible permutation, such as by using probabilistic or historical data to select a subset of permutations that are likely to occur. In some embodiments, an index may contain every practically possible, mathematically possible, or historically known permutation with respect to a certain species or group of species, such that permutations that will likely not be necessary to compress or decompress genomic information for a certain species or group of species may not be included in an index. In some embodiments, an index may not include permutations that are not known to occur in nature.

In some embodiments, the elements of an index may each be 16 bases in length and 128 bits in size, while the reference numbers may each be 8 bits in size. In some embodiments, the elements may be more or less than 16 bases in length and may be more or less than 128 bits in size. In some other embodiments, the elements may be shorter or longer, which will affect the overall size of each index, and will affect the number of elements that are necessary to represent a given sequence of a certain length. For example, in some embodiments, the elements may each be fewer than 16 bases in length, such as 12 or fewer bases in length, or 8 or fewer bases in length. In some embodiments, the elements may each be more than 16 bases in length, such as 20 or more bases in length, 24 or more bases in length, or 32 bases in length. Using bases comprising more or fewer bases affects the overall size of the index by affecting the size of each element and also the number of permutations 4^{k} that may be included in the index. An important consideration in choosing the number of bases in each index may be the overall storage capacity required to store an index comprised of bases of the chosen length; indexes of bases of a greater length may be require greater storage capacity.

In some other embodiments the elements may be comprised of more or less than four unique nucleotides. For example, some elements may contain a fifth wildcard base in addition to the four nucleotides A, T, C, and G. In such embodiments, 5^{k} elements (as opposed to only 4^{k} elements) are needed in order for an index to represent an exhaustive listing of all possible elements of length k. With elements of length 16, this would increase the number of elements from 4,294,967,296 to 152,587,890,625, representing about a 40-fold increase. With approximately 40 times more elements in such an index, approximately 40 times as much memory could be needed to accommodate such an index, and processing times for searching and navigating such an index could also be slowed.

In some embodiments, an index may be provided by way of physical transportation, such as being provided in a hard drive or in any other suitable computer memory. In some embodiments, an index may be provided by way of wired or wireless network communication, such as transmission over a private network or over the internet. In some embodiments, an index may be built on the computer (e.g., by engine 150) on which it resides. For example, a program, application, or other computer instructions may be provided to a computer, allowing the computer to construct the index and store it. For example, an algorithm may be provided as part of a computer program that is provided over the internet, and the algorithm may enable a computer to form and store an index.

In some embodiments, more than one index may be provided in the same computer system or at the same location or to the same party. For example, one index containing elements of length 16 may be provided, and another index containing elements of length 12 may be provided. In some embodiments, one index may contain both elements of length 16 and of length 12, or of any two or more element lengths k₁, k₂, etc. In some embodiments, such an index may be capable of compressing and/or decompressing genomic information with respect to a compression method using elements of length k₁, k₂, kₙ, etc., or any combination thereof. In some embodiments, an index may include multiple sets of reference numbers that allow the index to function as if it were an index containing multiple sets of elements of different lengths k. For example, an index containing 4¹⁶ elements of length 16 may contain every mathematically possible permutation of elements with 16 bases where the bases are either A, G, T, or C. That exhaustive set of 4¹⁶ bases may be understood, however, as itself containing the complete set all 4¹² mathematically possible permutations of elements of length 12 where the bases are either A, G, T, or C. By taking the first 12 bases (or any given contiguous portion of length 12) of each of the 16-base elements, for example, the leading 12 bases of 4¹² of the 4¹⁶ bases may account for an exhaustive set of all 4¹² mathematically possible permutations of elements of length 12. Thus, the 4¹² elements that account for the permutations of elements of length 12 may be assigned, in some embodiments, a second reference number that indicates an element's first 12 bases as being a given permutation. In this manner, by adding just 4¹² (under 17 million) reference numerals to an index having 4¹⁶ (over 4 billion) reference numerals and 4¹⁶ elements, the index may serve as two indexes for compressing and/or decompressing genomic information using elements of 16 and/or 12 bases in length.

Indexes that may be used in or with the systems, methods, and/or techniques described herein are described in U.S. patent application Ser. No. 13/904,738, titled “Systems and Methods for SNP Analysis and Genome Sequencing;” in U.S. patent application Ser. No. 14/718,950, titled “Compression and Transmission of Genomic Information;” and in U.S. patent application Ser. No. 15/977,659, titled “Primer Design Using Indexed Genomic Information;” each of which applications is hereby incorporated by reference in its entirety.

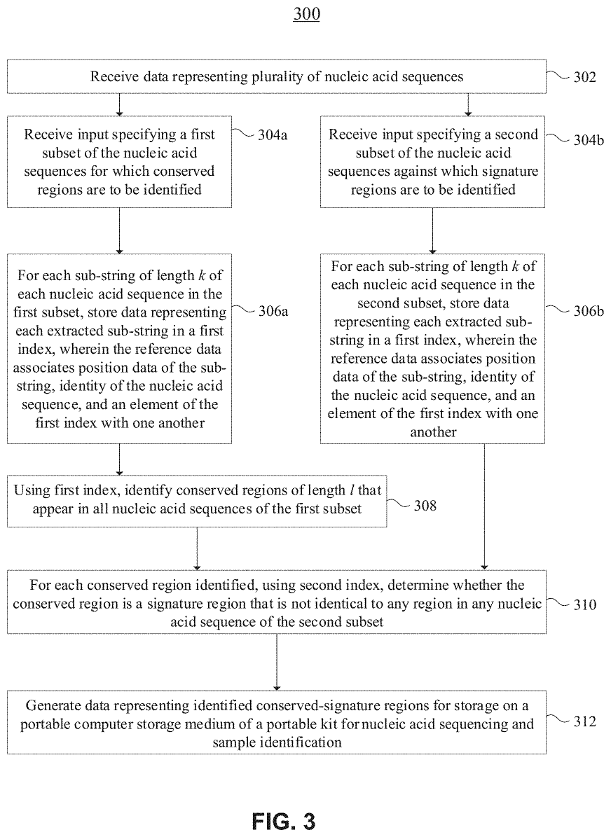

Engine 150 may be configured to use indexes—e.g., a first index corresponding to the first set of nucleic acid sequences, and a second index corresponding to the second set of nucleic acid sequences—to evaluate abundance criteria and sparsity criteria in order to identify conserved-signature sequences. The process of using indexes to assess said criteria is explained in additional detail below with reference to FIG. 3.

A sequence that is identified as a conserved-signature sequence may be transmitted from engine 150 to kit 101 for storage on storage medium 140, thereby configuring kit 101 for efficient and effective identification of target organisms in a specifically-contemplated deployment situation. By configuring kit 101 in this manner, kit 101 may be able to effectively and efficiently identify nucleic acid sequences associated with the target organism without need to have access to the entirety of database 160. Kit 101 may thus be used for sequence identification in situations in which network communication is not available or is ineffective.

In some embodiments, kit 101 may be loaded with conserved-signature sequences for a single organism. In some embodiments, kit 101 may be loaded with conserved-signature sequences for a plurality of organisms, such as a user-selected set of organisms and/or a predefined group of organisms. In some embodiments, kit 101 may be loaded with conserved-signature sequences for a predefined logical grouping of organisms, such as organisms that correspond to a specific type of organism or to specific trains. For example, kit 101 may be loaded with a “cassette” of signature-sequences for organisms classified as biological warfare agents, food pathogens, viruses, bacteria, fungi, mammalian, and/or harmful agents.

In some embodiments, once storage medium 140 has been loaded with a set of conserved-signature sequences, portable computing device 112 may then be used to perform one or more sample identification protocols using the stored conserved-signature sequences. In some embodiments, a nucleic acid sequence extracted from a collected sample—e.g., a sequence generated by sequencing system 110—may be compared to the stored conserved-signature sequences in order to determine one or more organisms, groups of organisms, and/or characteristics that are associated with the sample nucleic acid sequence. Determination of whether a sample nucleic acid sequence corresponds to a conserved-signature sequence stored on storage medium 140 may comprise determining whether the sequences are an exact match, a near match (e.g., above a threshold percentage of matching bases), and/or a probable match (e.g., a determination that the sample sequence is probably represented by a probabilistic data structure).

In some embodiments, determination of whether a sample nucleic acid sequence corresponds to a conserved-signature sequence stored on storage medium 140 may comprise generating one or more coverage metrics, such as calculating a number or percentage of sample sequences (e.g., sample k-mers) that correspond to a conserved-signature sequence for an organism, group of organism or characteristic; calculating a number or percentage of conserved-signature sequences for an organism that correspond to a sample sequence for a sample. In some embodiments, portable computing device 112 may generate a ranked list, based on calculated coverage metrics, showing which organisms (or groups of organisms or characteristics) correspond most strongly to sequences from a metagenomics sample, for example by ranking said organisms by number of matches and/or by percentage of matches.

In some embodiments, additionally or alternatively to storing conserved-signature sequences themselves on medium 140 of kit 101, a data structure representing the conserved-signature sequence may be stored on medium 140 of kit 101. For example, in some embodiments, one or more probabilistic data structures, such as bloom filters, representing a plurality of conserved-signature sequences as members of a set may be stored on medium 140 of kit 101. A probabilistic data structure representing a plurality of conserved-signature sequences as members of a set may be configured to be queried by a string (e.g., a k-mer from a sample nucleic acid sequence) to generate output data indicating whether the query string is (a) probably included in the set or (b) definitely not included in the set.

Probabilistic data structures may thus be used to encode conserved-signature sequence data on medium 140 of kit 101, such that one or more probabilistic data structures stored thereon may be used to quickly and efficiently perform comparisons between the conserved-signature sequences and the sample nucleic acid data. Furthermore, this encoding technique may allow for kit 101 to be deployed without publicly exposing the un-encoded signature-sequence information. Using probabilistic data structures to represent conserved-signature sequences on kit 101 may enable secure, fast, efficient, accurate, and precise transfer, storage, and analysis of sensitive secure-signature sequences, for example without exposing parties associated with the secure-signature sequences to unnecessary risk of being proven to be associated with the secure-signature sequences. A probabilistic data structure stored on the portable kit may confer the advantage that it may help to obfuscate the identity of one or more sequences represented by the data structure, which may be desirable in situations in which the conserved-signature sequences are proprietary and/or in which the conserved-signature sequences present security concerns. The methods may further allow for rapid and effective analysis of encoded secure-signature sequences and may further allow for efficient and compact transmission and storage of encoded secure-signature sequences, requiring less storage and processing resources than known methods.

Techniques for encoding, storing, transmitting, and/or analyzing genomic information using probabilistic data structures that may be used in or with the systems, methods, and/or techniques described herein are described in U.S. patent application Ser. No. 15/977,646, titled “Secure Communication Of Sensitive Genomic Information Using Probabilistic Data Structures,” and in U.S. patent application Ser. No. 15/977,667, titled “Rapid Genomic Sequence Classification Using Probabilistic Data Structures,” both of which applications are hereby incorporated by reference in their entirety.

In some embodiments, a first probabilistic data structure may represent sequences (e.g., k-mers) taken from each nucleic acid in the first set. In some embodiments, a probabilistic data structure may represent sequences from various reference sequences for the same organism, or various reference sequences for related organisms (e.g., organisms of different strains of the same species, or of similar species). In some embodiments, a probabilistic data structure may represent sequences (e.g., k-mers) from different organisms that share one or more sequence characteristics, or where the different organisms share one or more characteristics. In this manner, sets of nucleic acid sequences may be grouped into probabilistic data structures that represent phylogenic groupings or other logical groupings of organisms.

In some embodiments, a probabilistic data structure returning a result, when queried, indicating a probable match, may cause the system to generate an output indicating that a match has been determined. In some embodiments, an indication of a probable match may cause the system to automatically undertake further analysis of the sample sequence that was used to query the probabilistic data structure. For example, a deterministic comparison may be performed in response to a probable match being indicated.

In some embodiments, a probable match being indicated by one probabilistic data structure may cause the system to automatically perform an analysis using one or more additional probabilistic data structures. For example, in some embodiments, a plurality of probabilistic data structures may be arranged into a multi-level data structure, where probabilistic data structures in a lower level of the multi-level structure represent respective sets of conserved-signature sequences, and where probabilistic data structures in a higher level of the multi-level structure represent supersets (comprising multiple underlying sets) of probabilistic data structures. The probabilistic data structure may thus be organized into a logical tree pattern, such that a system may perform an initial query to determine whether a sample sequence (e.g., a sample k-mer) is included in a superset represented by a higher-level probabilistic data structure. If a positive result is returned, then the system may perform subsequent queries against each of the probabilistic data structures in the lower-level that underlie the initially-queried probabilistic data structure. In this way, the system may iteratively narrow down (over two or more levels of a multi-level data structure) the conserved-signature sequence set to which the sample sequence corresponds. (On the other hand, if the initial query of the higher-level probabilistic data structure returns a negative result, then processing may be stopped, such that processing resources and time are not unnecessarily spent querying each underlying probabilistic data structure when it is known that no match exists.) Additional processing of probable matches may thereafter be performed locally or remotely. For example, some processing may be performed on portable computing device 112, and instructions to perform additional processing (immediately or at a later time) may then responsively be transmitted to another device, such as to kit configuration engine 150 or to a cloud-hosted processing system.

In some embodiments, probabilistic data structures for storage on medium 140 may be generated by kit configuration engine 150 and transmitted to kit 101 before deployment of kit 101. In some embodiments, one or more aspects of a probabilistic data structure generated by engine 150 may be set in accordance with one or more user inputs, for example user inputs received from user device 170. For example, a user input may specify a set (or subset or superset) or sequences to be represented by a probabilistic data structure, a length of sequences (e.g., a length of k-mers) to be used for conserved-signature sequences represented in a probabilistic data structure, a number of sequences represented in a probabilistic data structure, a data size of a probabilistic data structure, a false-positive result rate of a probabilistic data structure, metadata associated with a probabilistic data structure, a relationship (e.g., a hierarchical relationship) of a probabilistic data structure to other probabilistic data structures within a multi-level data structure, and/or one or more automated actions to be triggered by a positive result returned by querying of a probabilistic data structure.

In some embodiments, additionally or alternatively to storing a probabilistic data structure on medium 140 of kit 101 to represent one or more conserved-signature sequences, an index data structure representing the conserved-signature sequence may be stored on medium 140 of kit 101. For example, an index data structure such as those described with reference to FIG. 2 may be stored on medium 140 of kit 101, wherein the index includes data indicating a conserved-signature sequence identity, location information for the conserved signature sequence, and an element/permutation of the index that represents the k-mer appearing in the conserved-signature sequence at that location. A sample identification protocol for a sample sequence may then include k-merizing the sample sequence and determining whether any data is stored in the index in association with the index element representing the sample k-mer.

In some embodiments, results of a sample identification protocol (e.g., an identity of a matching organism, a ranked list of matching organisms, etc.) may be stored locally on kit 101, may be displayed to a user, and/or may be transmitted from kit 101 to one or more other devices or other systems. In some embodiments, results of a sample identification protocol may be used to selectively trigger one or more automated actions, such as a DNA extraction protocol, a DNA sequencing preparation protocol, a sequencing protocol, and/or further sample identification protocols.

In some embodiments, results of a sample identification protocol (e.g., an identity of a matching organism, a ranked list of matching organisms, etc.) may be transmitted from portable computing device 112 to kit configuration engine 150, and the results of the protocol may be used to inform configuration of portable computing device 112 (or other similar devices) for subsequent deployments and/or subsequent use cases. For example, if it is determined by a sample identification protocol that a certain organism is likely to be present in samples in a certain environment, then this knowledge may be used to determine whether to use sequences corresponding to that organism a member of a set (e.g., a “second set” as contemplated above) when identifying conserved-signature sequences for future deployments of portable sequencing and sample identification kits.

As stated above, in addition to portable computing device 112 and portable computer storage medium 140, kit 101 may additionally include DNA extraction system 104, DNA sequence preparation system 106, and sequencing system 110.

In some embodiments, the different systems included in the kit include sub-components such as tools and equipment configured for field sequencing operations. In some embodiments, the DNA extraction system may include DNA extraction tools, a mini centrifuge, a high-G spin centrifuge, and a mixer configured to perform one or more steps of a DNA extraction protocol of the DNA extraction system. In some embodiments, performing the DNA extraction protocol yields extracted DNA from metagenomics sample. In some embodiments, the extracted DNA may be quantified by the fluorometer. In some embodiments, the extracted DNA may be prepared for sequencing via the sequencing preparation system. The sequencing preparation system may include DNA preparation tools and may include a sample preparation device for automating pipetting and manipulation steps of the sequencing preparation system. In some embodiments, the sequencing preparation system may include the mini centrifuge and the mixer. The prepared extracted DNA may then be sequenced via the sequencing system.

In some embodiments, kit 101 may be used for extracting DNA via DNA extraction system 104, quantifying the extracted DNA via fluorometer 108, preparing extracted DNA for sequencing via sequencing preparation system 106, and sequencing the extracted DNA via sequencer system 110. In some embodiments, kit 101 may include instructions for doing performing one or more of said operations, including instructions (e.g., code) for causing (e.g., by portable computing device 112) one or more electronic components to automatically carry out one or more of said operations.

As shown in FIG. 1, according to some embodiments, kit 101 may include components configured to extract DNA from a sample, prepare extracted DNA for DNA sequencing, and sequence the prepared extracted DNA. In DNA extraction system 104 may be configured to receive a sample containing DNA and output DNA extracted from the sample. In some embodiments, fluorometer 108 may be configured to quantify a concentration of the extracted DNA. In some embodiments, sequencing preparation system 106 may be configured to receive the extracted DNA and prepare the extracted DNA for sequencing. In some embodiments, sequencing system 110 may be configured to receive the prepared DNA and output sequencing information of the prepared DNA (e.g., by generating one or more nucleic acid sequences representing the sequenced DNA). In some embodiments, sequencing system 110 may include a sequencer device 128 and a heater 130 housed within an insulated casing 132.

In some embodiments, kit 101 may include one or more power supplies (e.g., supply 114), which may optionally be provided as integrated with or separate from portable computing device 112. In some embodiments, portable power supply 114 may be a battery of portable computing device 112, such that device 112 may power and control sequencing preparation device 126 and sequencer device 128. In some embodiments, portable power supply 114 may be configured to power first centrifuge 118, second centrifuge 120, mixer 122, fluorometer 108, and portable computing device 112.

In some embodiments, kit 101 may include a portable for enclosure 102 for housing components of the kit.

In some embodiments, kit 101 may be configured for processing RNA. For example, kit 101 may include an RNA extraction system and an RNA sequencing preparation system. The components of the RNA extraction system and the RNA sequencing preparation system may be respectively similar to the DNA extraction system and the DNA sequencing preparation system described herein, sharing one or more characteristics in common therewith. In some embodiments, RNA systems may include reagents configured for processing RNA and DNA systems may include reagents configured for processing DNA.

### DNA Extraction

In some embodiments, the DNA extraction system 104 may be configured to extract DNA from a sample using DNA extraction tools 116 and equipment 118, 120, 122. The sample may be collected, for example, from soil, compost, manure, water, cell samples, or the like. The sample may contain high humic acid content. The DNA extraction system 104 may be configured to remove factors in the sample that prevent efficient DNA amplification and high-quality DNA purification. In some embodiments, the DNA extraction tools may be a DNeasy PowerSoil Pro Kit.

In some embodiments, the DNA extraction system 104 may be configured to perform a DNA extraction protocol for extracting DNA that includes sample preparation, cell lysis, inhibitor removal, DNA binding, DNA washing, and DNA elution. In some embodiments, the DNA extraction protocol includes utilization of DNA extraction tools 116, the first centrifuge 118, the second centrifuge 120, and the mixer 122. In some embodiments, the DNA extraction tools 116 may include a plurality of reagents, each reagent may be configured to facilitate or carry out one or more steps of the DNA extraction protocol. In some embodiments, the DNA extraction tools 116 may include a plurality of tubes for containing the sample. In some embodiments, the DNA extraction tools 116 include one or more spin columns. In some embodiments the reagents may be added in a pre-determined sequence to the sample for extracting DNA.

In some embodiments, the first centrifuge may be configured to apply a high G-force to collected samples as part of the DNA extraction system protocol. The high G-force is used to separate contents within a processing tube (such as an Eppendorf tube). In some embodiments, the first centrifuge may have a small footprint and a quiet operation with or without a rotor lid. In some embodiments, the first centrifuge may have a rotor lid configured for fast and ergonomic lid locking. In some embodiments, the first centrifuge may automatically enter into an energy conservative mode after a predetermined amount of time of non-use. The energy conservative mode may reduce energy consumption and extend lifetime of the first centrifuge. In some embodiments, the predetermined amount of time may be at least 4 hours, 6 hours, or 8 hours. In some embodiments, the predetermined amount of time may be at most 14 hours, 12 hours, or 10 hours. In some embodiments, the predetermined amount of time may be 4-14 hours, 6-12 hours, or 8-10 hours.

In some embodiments, the first centrifuge may include a rotor configured to house a plurality of processing tubes. In some embodiments, the rotor may have a maximum rotor capacity of at least 12×1.5/2.0 mL tubes, 14×1.5/2.0 mL tubes, 16×1.5/2.0 mL tubes, 18×1.5/2.0 mL tubes. In some embodiments, the rotor may have a maximum rotor capacity of at most 26×1.5/2.0 mL tubes, 24×1.5/2.0 mL tubes, 22×1.5/2.0 mL tubes, 20×1.5/2.0 mL tubes. In some embodiments, the rotor may have a maximum rotor capacity of a 12-26×1.5/2.0 mL tubes, 14-24×1.5/2.0 mL tubes, 16-22×1.5/2.0 mL tubes, 18-20×1.5/2.0 mL tubes.

In some embodiments, the first centrifuge may have a maximum speed for quick separation. In some embodiments, the maximum speed for quick separation may be at least 10,000 RPM, 12,000 RPM, or 14,000 RPM. In some embodiments, the maximum speed for quick separation may be at most 20,000 RPM, 18,000 RPM, or 16,000 RPM. In some embodiments, the maximum speed for quick separation may be 10,000-20,000 RPM, 12,000-18,000 RPM, or 14,000-16,000 RPM.

In some embodiments, when powered by the portable power supply 114, the first centrifuge 118 may be configured to ramp to a target speed at a predetermined rate that enables the portable power supply 114 to deliver sufficient power for the ramping operation. For example, the predetermined ramping rate may be determined to allow the first centrifuge to increase power at a rate that does not overload the portable power supply 114. In some embodiments, the target speed may be the maximum speed of the first centrifuge.

In some embodiments, the predetermined rate may be slower than a predefined ramp rate preset by a manufacturer of the first centrifuge for line power use (e.g., laboratory use). For example, when the first centrifuge is powered by the portable power supply 114, the minimum programmable ramp preset by the manufacturer of the first centrifuge 118 may overload the portable power supply 114. However, when the first centrifuge 118 is powered by the portable power supply 114, ramping the first centrifuge 118 at the predetermined (e.g., more gradual) rate does not overload the portable power supply 114. In some embodiments, the predetermined rate may include ramping to an initial speed and then ramping from the initial speed to the target speed in one or more increments spaced apart from one another by one or more predetermined intervals. In some embodiments, the initial speed may be at least 1,000 RPM, 1,500 RPM, 2,000 RPM, 2,500 RPM, or 3,000 RPM. In some embodiments, the initial speed may be at most 6,000 RPM, 5,500 RPM, 5,000 RPM, 4,500. RPM, or 4,000 RPM. In some embodiments, the initial speed may be 1,000-6,000 RPM, 1,500-5,500 RPM, 2,000-5,000 RPM, 2,500-4,500 RPM, or 3,000-4,000 RPM. In some embodiments, the increments may be at least 200 RPM, 300 RPM, 400 RPM, or 500 RPM. In some embodiments, the increments may be at most 900 RPM, 800 RPM, 700 RPM, or 600 RPM. In some embodiments, the increments may be 200-900 RPM, 300-800 RPM, 400-700 RPM, or 500-600 RPM.

In some embodiments, the first centrifuge 118 may be configured to control an internal temperature of the first centrifuge 118. The first centrifuge 118 may also be configured to include a quick pre-cooling function inside the rotor. In some embodiments, the first centrifuge 118 may be configured to control the internal temperature of the first centrifuge 118 to at least 0° C., 5° C., 10° C., or 15° C. In some embodiments, the first centrifuge 118 may be configured to control the internal temperature of the first centrifuge 118 to at most 70° C., 60° C., 40° C., or 30° C. In some embodiments, the first centrifuge 118 may be configure to control the internal temperature of the first centrifuge 118 to 0-70° C.,

5-60° C., 10-40° C., or 15-30° C.

In some embodiments, the first centrifuge 118 may be configured to control the internal temperature of the first centrifuge 118 to at least 2° C., 3° C., or 4° C. at a maximum rotation speed of the first centrifuge 118. In some embodiments, the first centrifuge 118 may be configured to control the internal temperature of the first centrifuge 118 to at most 7° C., 6° C., or 5° C. at a maximum rotation speed of the first centrifuge 118. In some embodiments, the first centrifuge 118 may be configured to control the internal temperature of the first centrifuge 118 to 2-7° C., 3-6° C., or 4-5° C. at a maximum rotation speed of the first centrifuge 118.

In some embodiments, the first centrifuge 118 may have a low access height of at least 16 cm, 18 cm, 20 cm, or 22 cm. In some embodiments, the first centrifuge may have a low access height of at most 32 cm, 30 cm, 28 cm, or 26 cm. In some embodiments, the first centrifuge may have a low access height of 16-32 cm, 18-30 cm, 20-28 cm, or 22-26 cm.

In some embodiments, the second centrifuge 120 may be a miniaturized centrifuge (e.g., any centrifuge smaller and/or less powerful than the first centrifuge) configured to push liquid reagent stuck to the side of the processing tube towards a bottom of the tube via a spin protocol as part of one or more steps of the DNA extraction system protocol. Compared to a full-sized centrifuge, second centrifuge 120 may have a compact size and may be easier to operate for spinning samples for short spin times. Second centrifuge 120 may be preferred over a full-sized centrifuge for implementing a spin protocol that includes short spin time and does not require a specific g-force. In some embodiments, the short spin time may be at least 1 second or 2 seconds. In some embodiments, the short spin time may be at most 10 seconds, 8 seconds, 5 seconds, or 3 seconds. In some embodiments, the short spin time may be 1-10 seconds, 1-8 seconds, 1-5 seconds, or 1-3 seconds.

In some embodiments, the second centrifuge 120 may be a microfuge. In some embodiments, the second centrifuge 120 may include dynamic braking system. In some embodiments, the second centrifuge 120 may include a direct drive system and/or a vibration drive system. In some embodiments, the second centrifuge 120 may have an automatic speed control. In some embodiments, the second centrifuge 120 may be configured to operate at a constant speed. In some embodiments, the second centrifuge 120 may be configured operate on AC 85V-250V 1 A 50/60 Hz.

In some embodiments, the maximum revolutions per minute (RPM) of the second centrifuge 120 may be at least 5,000 RPM, 6,000 RPM, 7,000 RPM, or 8,000 RPM. In some embodiments, the maximum RPM of the second centrifuge 120 may be at most 10,000 RPM, 9,000 RPM, 8,000 RPM, 7,000 RPM, or 6,000 RPM. In some embodiments, the maximum RPM may be 5,000 RPM-10,000 RPM, 6,000 RPM-9,000 RPM, or 7,000 RPM-8,000 RPM.

In some embodiments, the relative centrifugal force (RCF) of the second centrifuge 120 may be at least 600×g, 700×g, or 800×g. In some embodiments, the RCF of the microfuge may be at most 4,500×g, 4,500×g, or 3,500×g. In some embodiments, the RCF of the microfuge may be 600-4,500×g, 700-4,000 g, or 800-3,500×g.

In some embodiments, the second centrifuge 120 may be configured to accelerate and decelerate. In some embodiments, the second centrifuge 120 may be configured to accelerate to 90% of a rated speed at least within 3 seconds, 4 seconds, or 5 seconds. In some embodiments, the second centrifuge 120 may be configured to accelerate to 90% of a rated speed at most within 7 seconds, 6 seconds, or 5 seconds. In some embodiments, the second centrifuge 120 may be configured to accelerate to 90% of a rated speed within 3-7 seconds, or 4-6 seconds.

In some embodiments, the deceleration of the second centrifuge 120 is based on whether a cover of the second centrifuge 120 is open or closed. In some embodiments, if the cover is open, the second centrifuge 120 may be configured to decelerate at least within 1 second, 2 seconds, or 3 seconds. In some embodiments, if the cover is open, the second centrifuge 120 may be configured to decelerate at most within 6 seconds, 5 seconds, or 4 seconds. In some embodiments, if the cover is open, the second centrifuge 120 may decelerate within 1-6 seconds, 2-5 seconds, or 3-4 seconds.

In some embodiments, if the cover is closed, the second centrifuge 120 may be configured decelerate at least within 10 seconds, 12 seconds, or 14 seconds. In some embodiments, if the cover is closed, the second centrifuge 120 may be configured to decelerate at most within 24 seconds, 22 seconds, or 20 seconds. In some embodiments, if the cover is closed, the second centrifuge 120 may be configured to decelerate within 10-24 seconds, 12-22 seconds, or 14-20 seconds.

In some embodiments, the mixer 122 may be part of one or more steps of the DNA extraction system protocol. In some embodiments, the mixer 122 may be a compact vortex mixer (also known as a vortexer) configured to agitate reagents and ensure homogenous mixing of reagents within processing tubes (such as Eppendorf vials). In some embodiments, electrical conditions of the mixer 122 may include 100 to 240V and 50/60 Hz. In some embodiments, the mixer 122 may have a speed range of 0-4,000 RPM, 0-3,000 RPM, or 0-2,000 RPM.

In some embodiments, the mixer 122 may have a portable size. In some embodiments, the mixer 122 may have a height of at least 1 inches, 2 inches, or 3 inches. In some embodiments, the mixer 122 may have a height of at most 7 inches, 6 inches, of 5 inches. In some embodiments, the mixer 122 may have a height 1-7 inches, 2-6 inches, or 3-5 inches. In some embodiments, the mixer 122 may have a length of at least 6 inches, 7 inches, or 8 inches. In some embodiments, the mixer 122 may have a length of at most 11 inches, 10 inches, or 9 inches. In some embodiments, the mixer 122 may have a length of 6-11 inches, 7-10 inches, or 8-9 inches. In some embodiments, the mixer 122 may have a width of at least 4 inches, 5 inches, or 6 inches. In some embodiments, the mixer 122 may have a width of at most 9 inches, 8 inches, or 7 inches. In some embodiments, the mixer 122 may have a width of 4-9 inches, 5-8 inches, or 6-7 inches.

In some embodiments, the mixer 122 may have a load bearing capacity of at least 0.5 lb or 1 lb. In some embodiments, the mixer 122 may have a load bearing capacity of at most 3.5 lbs or 2.5 lbs. In some embodiments, the mixer 122 may have a load bearing capacity of 0.5-3.5 lbs or 1-2.5 lbs.

In some embodiments, the extracted DNA of the DNA extraction system 104 is purified DNA and may be configured to make DNA of the sample accessible and available for one or more downstream processes such as quantification and sequencing.

### DNA Quantification

In some embodiments, a fluorometer 108 may be used to quantify a concentration of eluted DNA from the DNA extraction system 104. In some embodiments, a portion of the eluted DNA may be used for the quantification, and in some embodiments that portion cannot thereafter be sequenced. In some embodiments, the fluorometer 108 may be have a small footprint suitable for a portable fluorometer. In some embodiments, the fluorometer may be a Qubit 4 fluorometer.

In some embodiments, the fluorometer 108 may quantify DNA, RNA, and protein at least within 1 second, 2 seconds, 3 seconds, or 4 seconds. In some embodiments, the fluorometer 108 may quantify DNA, RNA, and protein at most within 9 seconds, 8 seconds, 7 seconds, or 6 seconds. In some embodiments, the fluorometer 108 may quantify DNA, RNA, and protein within 1-9 seconds, 2-8 seconds, 3-7 seconds, or 4-6 seconds. In some embodiments, the fluorometer 108 measures intact RNA within at least 2 seconds, 3 seconds, 4 seconds, or 5 seconds. In some embodiments, the fluorometer 108 measures intact RNA within at most 9 seconds, 8 seconds, 7 seconds, or 6 seconds. In some embodiments, the fluorometer 108 measures intact RNA within at most 2-9 seconds, 3-8 seconds, 4-7 seconds, or 5-6 seconds.

In some embodiments, the fluorometer 108 may quantify DNA with small volumes or very dilute samples. In some embodiments, a small volume may be at least 0.5 μL or 1 μL. In some embodiments, a small volume may be at most 40 μL, 30 μL, 20 μL. In some embodiments, a small volume may be 0.5-40 μL, 1-30 μL, or 1-20 μL.

In some embodiments, data associated with DNA quantification may be stored on the fluorometer 108 for at least up to 250 samples, 500 samples, or 750 samples. In some embodiments, the data associated with DNA quantification may be stored on the fluorometer 108 for at most up to 2,000 samples, 1,500 samples, or 1,000 samples. In some embodiments, the data associated with DNA quantification may be stored on the fluorometer 108 for up to 250-2,000 sample, 500-1,500 samples, or 750-1,000 samples.

In some embodiments, the fluorometer 108 may include a user interface that has a color touch screen. The user interface may display graphical data associated with quantification of samples when samples are quantified to be in a predetermined range. In some embodiments, the fluorometer 108 may display content in a plurality of languages such as English, French, Spanish, Italian, German, simplified Chinese, and Japanese. In some embodiments, data from the fluorometer 108 may exported via a WIFI dongle, a USB drive, or via a USB cable.

The fluorometer 108 may be programmable to run assays for DNA quantification. In some embodiments, the fluorometer 108 may include pre-programmed assays. In some embodiments, the fluorometer 108 may be configured to allow a user to program their own assay.

### Sequencing Preparation

In some embodiments, the eluted DNA of the extraction system 104 may be processed by the sequencing preparation system 106. In some embodiments, the sequencing preparation system 106 is configured to perform a preparation protocol for generating sequencing libraries from the extracted DNA of the extraction system 104. The preparation protocol may include utilization of the DNA sequencing tools 124, the second centrifuge 120, the mixer 122, and the sample preparation device 126 of the sequencing preparation system 106. In some embodiments, the DNA sequencing tools 124 may include a plurality of reagents, one or more of which may be configured to facilitate one or more steps of the DNA preparation protocol. In some embodiments, the DNA sequencing tools may be a Rapid Sequencing Kit. In some embodiments, the preparation protocol may be a two-step protocol that involves cleaving the eluted DNA molecules and attaching tags to the cleaved ends, and then adding sequence adapters to the tagged ends. In some embodiments, the cleavage is transposase-based. In some embodiments, a read length is a random distribution based on an input fragment length.

In some embodiments, a preparation time of the preparation protocol may be rapid. In some embodiments, the preparation time may be at least 6 minutes, 8 minutes, or 10 minutes. In some embodiments, the preparation time may be at most 16 minutes, 14 minutes, of 12 minutes. In some embodiments the preparation time may be 6-16 minutes, 8-14 minutes, or 10-12 minutes.

In some embodiments, the sequencing preparation system 106 may be configured to process extracted DNA that has or has not undergone polymerase chain reaction (PCR). In some embodiments, if the extracted DNA does not contain a sufficient amount of genetic material for DNA sequencing, then kit 101 may be used to perform PCR. In some embodiments, the sequencing preparation system 106 may process a minimum of 200 ng, 300 ng, or 400 ng of high molecular weight DNA from the extraction system 104. In some embodiments, the sequencing preparation system 106 may process at most 700 ng, 500 ng, or 600 ng of high molecular weight DNA from the extraction system 104. In some embodiments, the sequencing preparation system 106 may process 200-700 ng, 300-500 ng, or 400-600 ng of high molecular weight DNA from the extraction system 104.

In some embodiments, the sample preparation device 126 may be configured to automate one or more protocol steps associated with preparing a sequencing library. Preparation device 126 may be configured to perform a final step of sample preparation (e.g., before sequencing). The protocol may be predefined or a custom sample preparation protocol defined by a user. In some embodiments, the sample preparation device 126 may be configured to automate one or more steps of the sample preparation protocol of the sequencing preparation system 106. For example, the sample preparation device 126 may be configured to replace manual pipetting and manual manipulation steps (such as mixing and separation between reagents) of the sample preparation protocol. The automated sample preparation device 126 may be configured to reduce hands-on sample preparation time and achieve a high level of reproducibility for library preparations.

In some embodiments, the sample preparation device 126 may be a portable, lightweight, robust automated device configured to provide a controlled environment for incubations and extractions. In some embodiments, the sample preparation device 126 may include a metal chassis that includes a heater, a peltier, an optical fluorescent detector and magnets. In some embodiments, the sample preparation device 126 may be a Voltrax V2 device.

In some embodiments, the DNA sequencing preparation system 106 outputs prepared DNA based on the eluted DNA. The prepared DNA may be configured for processing by the sequencing system 110. For example, the prepared DNA may include attachments (such as transposase and sequencing adapted molecules) that help guide the DNA for sequencing by the sequencing system 110.

### Sequencing

In some embodiments, the sequencer device 128 of the sequencing system 110 may be configured to receive the prepared DNA of the DNA sequencing preparation system 106. In some embodiments, the sequencer device 128 performs direct sequencing of the prepared DNA and outputs sequencing information. In some embodiments, the sequencer device 128 may be a MinION.

In some embodiments, the sequencer 128 may include a sample intake, a consumable flow cell, a sensor array, a sensor chip (Application-Specific Integrated Circuit, ASIC), and a USB port. In some embodiments, the sample may be loaded into the sequencer 128 via the sample intake. In some embodiments, the sample may flow through the consumable flow cell and interface with electronics of the sequencer 128. In some embodiments, the sensor array may include a plurality of sensors communicatively connected to a plurality of electrodes. In some embodiments, the plurality of electrodes may be connected to a plurality of channels of a sensor chip (ASIC). The individual channels of the sensor chip (ASIC) may acquire, measure, and control data from the sensor array.

In some embodiments, the USB port of the sequencer device 128 may be configured to connect the sequencer to a portable computing device, a computer, or other device such that the portable computing device, computer, or other device may operate the sequencer device 128.

In some embodiments, the sequencer device 128 may include an onboard heater configured to maintain an operational temperature. In some embodiments, the onboard heater may not be sufficient to maintain the operational temperature in cold conditions, for example, when the air temperature surrounding the sequencer device 128 is less than or equal to about 2° C. In some embodiments, the sequencing system 110 may include an external heater 130 and an insulated housing 132 configured to maintain the operational temperature of the sequencer device 128 when the air temperature is less than or equal to about 2° C.

In some embodiments, the insulated housing 132 may be configured to enclose the sequencer device 128 and the external heater 130. In some embodiments, the insulated housing 132 may have dimensions of at least 9 inches×3 inches×2 inches. In some embodiments, the insulated housing 132 may have dimensions of at most 14 inches×9 inches×8 inches. In some embodiments, the insulated housing 132 may have dimensions of 9-14 inches×3-9 inches×2-8 inches. In some embodiments, the insulated box may be made from Styrofoam.

In some embodiments, the external heater may be a heatwrap positioned about, adjacent, or at a predetermined distance from the sequencer device 128.

### Portable Computing Device

In some embodiments, the portable computing device 112 may perform computer processing for one or more components of the kit, and may provide power to one or more components from an on-board power supply separate from the external power supply (power supply 114) discussed below. In some embodiments, the portable computing device 112 may be a consumer-grade laptop or tablet device with one or more USB ports for connecting to kit components. For example, the portable computing device 112 may be configured to power and control the sequencer device 128 and the sample preparation device 126 via one or more USB ports. In some embodiments, the portable computing device 112 may be a consumer-grade laptop or tablet.

The portable computing device 112 may be configured to operate software for sequencing, basecalling, and organism identification. For example, the portable computing device 112 may include offline-MinKnow for sequencing, Guppy for basecalling, and Centrifuge for organism identification using a pre-computed reference database.

### Power Supply

In some embodiments, the portable power supply 114 may be configured to power the first centrifuge 118, the second centrifuge 120, the mixer 122, the fluorometer 108, and the portable computing device 112. In some embodiments the power supply 114 may be a PLUG portable power supply. In some embodiments, the power supply 114 may have a battery capacity of at least 30,000 mAh, 40,000 mAh, or 50,000 mAh. In some embodiments, the battery capacity may be at most, 80,000 mAh, 70,000 mAh, or 60,000 mAh. In some embodiments, the battery capacity may be 30,000-80,000 mAh, 40,000-70,000 mAh, or 50,000-60,000 mAh. In some embodiments, the portable power supply 114 may include a charging port, two international AC outlets, two fast charge USB ports, and one USB type C port. In some embodiments, the power supply 114 may be configured to have 110 V and 220V options.

In some embodiments, the portable power supply 114 may be configured to have dimension of at least 3 inches×6 inches×0.5 inches. In some embodiments, the portable power supply 114 may be configured to have dimension of at most 7 inches×10 inches×3 inches. In some embodiments, the portable power supply 114 may be configured to have dimension of 3-7 inches×6-10 inches×0.5-3 inches.

In some embodiments, one or more portable power supplies may be configured to power equipment in the field and the portable enclosure 102 may be configured to house the one or more portable power supplies.

Portable kits such that may be used in or with the systems, methods, and/or techniques described herein are described in U.S. patent application Ser. No. 17/231,280, titled “Portable Field-Deployable Nucleic Acid Sequencing Kit,” which application is hereby incorporated by reference in its entirety.

FIG. 3 shows a method 300 for configuring a portable nucleic acid sequencing and sample identification kit, in accordance with some embodiments. In some embodiments, method 300 may be performed by a system comprising one or more processors in order to generate conserved-signature sequences for storage on (or representation on) a portable sequencing and sample identification kit. In the example of FIG. 1, method 300 may be performed by kit configuration engine 150 in order to identify conserved-signature sequences for storage on portable computer storage medium 140 of kit 101.

At block 302, in some embodiments, the system may receive data representing a plurality of nucleic acid sequences. In some embodiments, the system receiving the data may be any computer system capable of receiving, storing, and processing data representing a plurality of nucleic acid sequences, such as system 100 of FIG. 1. The data may be received in any suitable manner, including receiving the information over a computerized communication medium (e.g., network communication, communication with physical storage media, manual entry, etc.) and including deriving and/or aligning the information directly (e.g., the input data may be extracted and/or aligned by the same computer that compresses the input data).

The data representing a plurality of nucleic acid sequences may be in any one or more suitable and readable formats, and may represent any portion of various genomic sequences, including one or more complete genomic sequence (e.g., Whole Genome Sequencing data, or “WGS” data). The data may be expressed in any suitable language or character encoding scheme, including, for example, ASCII or UTF-8.

In some embodiments, the data may represent a large body of nucleic acid sequences, such as hundreds of thousands or millions of nucleic acid sequences, taken from a database or database of genomic information. In some embodiments, the data may be received from one or more public databases, such as databases accessible via the internet, such as a National Center for Biotechnology Information (NCBI) database; in some embodiments, one or more private databases or sources of genomic information may alternately or additionally be used.

In some embodiments, the data may include metadata associated with one or more of the nucleic acid sequences, and such metadata may identify an organism or organisms associated with a nucleic acid sequence (e.g., the metadata may identify the nucleic acid sequences).

In some embodiments, the data may or may not be divided into one or more subsequences of predefined length (e.g., it may or may not be k-merized) before receipt by the system. In some embodiments, the receiving system may divide (all or some of) the received data into one or more subsequences of predefined length (e.g., k-merize the received data) following receipt.

In some embodiments, the data may be provided to the system as part of a manual upload from a user, as part of a batch download, or via access by the system to one or more libraries or databases of nucleic acid sequence data. In the example of system 100 of FIG. 1, engine 150 may receive data representing a plurality of nucleic acid sequences by receiving data from (and/or receiving access to all or some of the data stored on) reference nucleic acid database 160.

At block 304a, in some embodiments, the system may receive an input specifying a first subset of the nucleic acid sequences for which conserved regions are to be identified. As described above, the first user input may comprise an explicit or implicit indication of a set of one or more of the sequences received by or available to the system, wherein the indicated set of sequences are those sequences for which the portable kit will be configured to search. That is, the first subset of sequences may be understood to be the “target” subset of sequences, such that the kit will be configured to target (in sample identification protocols) one or more target organisms associated with the first subset of sequences. As described herein, the system may configure the kit to target the one or more organisms associated with the first set of sequences by identifying one or more sequences that are conserved regions appearing in every one (or in a sufficiently high percentage) of the sequences in the first subset.

In some embodiments, the user input specifying the first subset of the nucleic acid sequences for which conserved regions are to be identified may be provided to the system via one or more graphical user interfaces. For example, the system may display a graphical user interface for a user, and the user may be able to execute one or more inputs controlling the system via the graphical user interface. In some embodiments, the graphical user interface may be provided as an interface on a workstation that performs one or more of the other processing steps described herein; in some embodiments, the graphical user interface may be provided as a web-browser hosted interface, such that a user may control the processing steps described herein via the internet from a remote device, such as a personal computer, laptop, tablet, or smart phone.

In some embodiments, the user interface may be configured to accept the user input specifying the first subset of sequences along with one or more additional user inputs, such as other user inputs described herein. In some embodiments, other user inputs accepted by the user interface may include a user input specifying one or more of the following: one or more nucleic acid strings (e.g., bases constituting a nucleic acid sequence), an identifier of one or more nucleic acid sequences, an identity (e.g., a name) of one or more organisms associated with one or more nucleic acid sequences, an identity (e.g., a name) of one or more groups of organisms (such as an organism type (e.g., biological warfare agents, food pathogens, viruses, bacteria, fungi, mammalian, and/or harmful agents) and/or a phylogenic classification), a characteristic of one or more nucleic acid sequences (e.g., length, patterns of bases, etc.), and/or a trait (e.g., a phenotype) of one or more organisms. In some embodiments, predefined organism types (e.g., biological warfare agents, food pathogens, viruses, bacteria, fungi, mammalian, and/or harmful agents) may be stored in association with various associated nucleic acid sequences, for example by associating one or more metadata tags with relevant sequences.

In some embodiments, the user interface may prompt a user to indicate what sequences, what organisms, and/or what kinds of organisms the user wishes to search for. The user may enter a responsive input by typing into a field and/or by selecting options from a drop-down or other menu.

In some embodiments, a user may indicate a plurality of identities, groups, characteristics, and/or traits for inclusion in the first subset. For example, a user may indicate that he wishes to configure the kit to search for a plurality of distinct organisms that may or may not bear any phylogenic or phenotypic relationship to one another.

Based on the user input specifying the first subset of sequences, the system may filter the plurality of nucleic acid sequences to which is has access, thereby selecting the first subset as those which satisfy any one or more criteria indicated by the user input.

In the example of system 100 of FIG. 1, engine 150 may receive the user input from user device 170, and may responsively select nucleic acid sequences from database 160 that comply with one or more criteria indicated by the received user input.

In some embodiments, the system may k-merize the selected subset of sequences by dividing them into sub-strings of length k.

At block 306a, in some embodiments, the system may, for each sub-string of length k of each nucleic acid sequence in the first subset, store data representing each extracted sub-string in a first index, wherein the reference data associates position data of the sub-string, identity of the nucleic acid sequence, and an element of the first index with one another. In some embodiments, the index in which the data is stored may share any one or more characteristics in common with index 200 described above with reference to FIG. 2.

In some embodiments, the process of storing data representing each sub-string in the first index may include building and/or seeding a first index with information regarding all of the nucleic acid sequences of the subset.

In some embodiments, the system may start with a first index stored on or otherwise accessible by the system (or the first index may be created/built in accordance with instructions accessible by the system), such as by being stored on memory 106. The first index may include a plurality of elements, where each element represents a permutation of nucleic acid bases of the same length k as the length k of the sub-strings. In some embodiments, the first index may have any or all of the properties described above with respect to index 200 of FIG. 2.

Initially, the first index may be a “blank-slate” index such as the index described above with reference to FIG. 2, in that it may not contain any location information such as reference numbers 204 and may not contain other information that is specifically related to a particular nucleic acid sequence. In accordance with the technique of block 308, the system may then seed the first index by inserting a plurality of data structures into the first index, wherein each data structure is associated with one of the sub-strings of a nucleic acid sequence of the subset. In some embodiments, the data structures inserted into the first index may share some or all characteristics in common with the reference numbers described above with respect to FIG. 2. In some embodiments, each data structure inserted into the first index may associate three or more pieces of information with one another: (1) the identity of the nucleic acid sequence of which the sub-string is a part; (2) the position in the nucleic acid sequence to which the sub-string corresponds; and (3) the element/permutation of the first index to which the sub-string corresponds. In some embodiments, the three pieces of information indicated above may be associated with one another by associated data stored in a data structure in the first index.

The data corresponding to the identity of the nucleic acid sequence may comprise any suitable metadata, such as the identification metadata described above.

The data corresponding to the position in the nucleic acid sequence to which the sub-string corresponds may comprise a number indicating a base at which the sub-string begins or ends in the nucleic acid sequence. Thus, for the first sub-string in a nucleic acid sequence, the position data saved to the first index may indicate position 1.

Finally, the data corresponding to the element/permutation of the first index to which the sub-string corresponds may comprise a reference to the element in the first index. In some embodiments, the stored reference comprises a pointer to the corresponding element in the first index (and/or the element may include a pointer to the stored reference). The pointer may, for example, be in the form of a reference number. In some embodiments, the reference may subsequently be used to look up the corresponding element in the first index, and may also be used to look up corresponding elements having the same reference number in other indexes (such as the second index described below). In some embodiments, the reference may comprise an 8-bit data structure, such as a single integer in ASCII or UTF-8. For example, the reference may be any one of the reference numbers 0 through 4^{k-1} shown in FIG. 2. In some embodiments, the reference may be a data structure of more than or fewer than 8 bits, for example 16 bits, 32 bits, 64 bits, or 128 bits, and may be stored along with, in association with, and/or with pointers to the data indicating the position and identity information discussed above.

In some embodiments, this process may be conceptualized as storing data representing sub-strings in various different “bins” of the first index, where each bin corresponds to a specific element of the first index representing a specific permutation of bases. For each sub-string, an indication of position and an indication of the identity of the overall nucleic acid sequence of which the sub-string is a part may be inserted into a bin that represents the same permutation of bases that constitute the sub-string.

Once all sub-strings for all nucleic acid sequences of the subset are seeded into the first index, the first index may contain multiple data structures representing sub-strings that correspond to the same element, indicating that more than one distinct sub-string extracted from the subset of nucleic acid sequences has the same permutation of 16 bases in the same order. Reference to this first index may thus facilitate the fast look-up of sub-strings in any of the nucleic acid sequences that have been seeded into the first index.

In the example of system 100 of FIG. 1, the first index may be created and/or configured by kit configuration engine 150, and the first index may be stored on any suitable electronic storage medium to which engine 150 has access.

At block 304b, in some embodiments, the system may receive an input specifying a second subset of the nucleic acid sequences against which signature regions are to be identified. Block 304b may share any one or more characteristics in common with block 304a. While the input(s) received at block 304a may indicate one or more criteria for selecting a first subset of nucleic acid sequences that the user intends to search for, the input(s) received at block 304b may indicate one or more criteria for selecting a second subset of nucleic acid sequences that the user intends to distinguish against. That is, nucleic acid sequences in the second subset may be those sequences that a user expects to be present in an environment of the target sequences, such that the system may select conserved-signature sequences that are present in the target sequences but that are not present in the sequences to be distinguished. In this manner, the second subset may represent those sequences that the user wishes to ensure do not create false positive results during sample-identification. In some embodiments, the second input(s) may indicate any one or more sequence identities, sequence characteristics, organism identities, organism groups, and/or organism characteristics, for example as described above with reference to the first input(s) at block 306a. In some embodiments, the second input(s) may be received via a user interface in any of the manners described above with reference to the first input(s) at block 306a. In the example of system 100 of FIG. 1, engine 150 may receive the user input from user device 170, and may responsively select nucleic acid sequences from database 160 that comply with one or more criteria indicated by the received user input.

At block 306b, in some embodiments, the system may, for each sub-string of length k of each nucleic acid sequence in the second subset, store data representing each extracted sub-string in a first index, wherein the reference data associates position data of the sub-string, identity of the nucleic acid sequence, and an element of the first index with one another.

Block 306b may share any one or more characteristics in common with block 306a. While block 306a concerns the creation and/or configuration of a first index representing the first subset of nucleic acid sequences (to be targeted), block 306b concerns the creation and/or configuration of a second index representing the second subset of nucleic acid sequences (to be distinguished).

In the example of system 100 of FIG. 1, the first index may be created and/or configured by kit configuration engine 150, and the first index may be stored on any suitable electronic storage medium to which engine 150 has access.

In some embodiments, additionally or alternatively to creating two separate indexes—a first one to represent those sequences to be targeted and a second one to represent those sequences to be distinguished—the system may create a single combined index that represents both the sequences to be targeted and the sequences to be distinguished. The system may, in some embodiments, create a combined index by storing data structures representing both the first and second subset of sequences in association with elements of a single combined index, wherein the data structures include additional metadata indicating whether they correspond to the first subset of sequences (to be targeted) or the second subset of sequences (to be distinguished). In some embodiments, using a combined index may require less memory than using two separate indexes.

At block 308, in some embodiments, the system may, using the first index, identify conserved regions of length l that appear in all nucleic acid sequences of the first subset. In some embodiments, in addition to or alternatively identifying conserved regions that appear in all nucleic acid sequences of the first subset, the system may identify conserved regions that appear in a minimum threshold percentage of nucleic acid sequences of the first set. Reference in the specification may be made to determining that a region is conserved for all nucleic acid sequences in a subset, but it is to be understood that the same techniques may be applied to ensure that a region is conserved for a threshold minimum percentage of nucleic acid sequences in a subset. (In some embodiments, the threshold minimum number of sequences may be configurable by a user input, for example by a user input received from a user of user device 170.) In the example of system 100 of FIG. 1, the processing steps of block 308 may be performed by kit configuration engine 150.

In some embodiments, once the first index has been created and seeded with data corresponding to the sub-strings from the first subset of nucleic acid sequences, the system may then determine which, if any, of the sub-strings are conserved across all of the nucleic acid sequences in the first subset. Thus, the system may analyze the data stored in the first index in order to search for identical sub-strings that appear in every nucleic acid sequence in the first subset. In some embodiments, the system may search for identical sub-strings that appear in any position of all of the nucleic acid sequences, while in some embodiments a system may search only for identical sub-strings that appear in a consistent, corresponding, or identical position in each of the nucleic acid sequences.

In some embodiments, the length l may be automatically determined by a system and/or may be manually settable and adjustable by a user. In some embodiments, the length l may be specified by one or more user inputs executed by a user of a user interface of device 170. In some embodiments, longer lengths l may yield fewer sub-strings that are conserved (e.g., common, identical, matching) across all members of the first subset, while they may be more likely to yield conserved sub-strings that are unique as compared to all known nucleic acid sequences outside the first subset (e.g., sequences in the second subset). In some embodiments, a specific length l, or a length l within a certain range, may be desirable for sample identification; for example, lengths l of longer than 25, 50, 75, 100, 125, 150, or 200 base pairs may be desirable, while lengths l of shorter than 250, 225, 200, 175, 150, or 125 base pairs may be desirable.

In some embodiments, this locating process may be include scanning down the length of any one of the nucleic acid sequences of the first subset in order to verify that it matches all other nucleic acid sequences in the first subset at each sub-string of length k; when a continuous common portion of minimum length l is located across all nucleic acid sequences in the first subset, then the system may determine that the portion is a conserved region. As compared to making this comparison on a base by base basis, using the first index to compare on a k-mer by k-mer basis may make this process significantly more fast and efficient. The scanning process is explained further below.

In some embodiments, for a given position in a first nucleic acid sequence of the first subset, it may be determined whether the data stored in the first index for that given position of the first nucleic acid sequence matches the data stored in the first index for the same element and for a corresponding position for every other nucleic acid sequence in the first subset (or for a sufficiently high percentage thereof). Any one of the nucleic acid sequences in the subset may be selected as the first nucleic acid sequence to start the comparison; in some embodiments, the longest nucleic acid sequence of the first subset may be selected, the nucleic acid sequence having the most reliable or highest quality data may be selected, or a user may manually choose (e.g., in accordance with input received from user device 170) one of the nucleic acid sequences in the first subset to serve as the first nucleic acid sequence.

The system may begin the determination by analyzing an initial position of the first nucleic acid sequence by determining whether data stored in the first index for the first nucleic acid sequence matches data stored in the first index for all other nucleic acid sequences in the first subset. In order to determine that a region is conserved, it may be required that the portions of the region be located in every one (or in a sufficiently high percentage) of the nucleic acid sequences in the first subset and in a corresponding (e.g., same or sufficiently similar) position in each nucleic acid sequence.

In some embodiments, the system may require that position data stored in the first index be matched across all sequences in the index in order for a matching k-mer to successfully be established and for the technique to proceed to block 316. However, matching or identical position data across all sequences in the index may not be required in all embodiments. For example, if all of the sequences in the first subset are complete genome WGS data, then conserved regions of sufficient length l may only be found at the same absolute biological position in the genome, and should therefore have identical position data for all complete genome sequences. However, if any of the nucleic acid sequences in the first subset are not a complete genome, and instead represent a portion of the genome starting at a different base than other sequences in the sub-set, then position data seeded into the index for each genome may not be identical for bases that correspond to the same absolute biological position in the genome. For example, position data may be shifted by five bases for a sequence in which the first five bases are missing. Furthermore, in some embodiments, a system may not require that position for conserved regions is common across different nucleic acid sequences at all, such that conserved region may be identified at for any portion of each of the nucleic acid sequences in the first subset.

In some such embodiments, the system may not require absolute matching of position data across all nucleic acid sequences in the first index. Instead, the system may ensure that position data for all sequences in the first subset adequately corresponds across the length of an entire conserved region l in order to establish that the same continuous identical string of l bases exists in each nucleic acid sequence. Requiring that the portions of the continuous region be located in a “corresponding” position in each nucleic acid sequence may simply require that each nucleic acid sequence has all bases of the continuous portion in the same order with respect to one another, while it may not require that the overall conserved region is located at the same absolute position in each nucleic acid sequence. In some embodiments, ensuring that conditions requiring corresponding positions are satisfied may simply require ensuring that each portion of a conserved region is offset from each other portion of a conserved region by the same number of bases in each nucleic acid sequence. Thus, ensuring that position data corresponds may include ensuring that position data for each k-mer included in a conserved region are set off from one another by the same number of bases across all nucleic acid sequences, even if the absolute position data stored in the first index indicates that the conserved region starts a different number of bases from the beginning of one or more of the nucleic acid sequences.

Alternately, in some embodiments, nucleic acid sequences of different length or starting at different portions in the genome may be accounted for by normalizing the position data for absolute biological position in the genome before storing position data to the index, such as by aligning partial nucleic acid sequences to a complete genome and using a common position convention (e.g., a convention geared to the complete genome) for position data in the first index.

In some other embodiments, however, a system may require that position data (e.g., absolute position data and/or the positon of a continuous region in the genome itself) match for each nucleic acid sequence, indicating that the same conserved portion is located at the same absolute biological position of each nucleic acid sequence. Thus, the system may in some embodiments search only for portions that are identical across all nucleic acid sequences at a common position in each of the nucleic acid sequences.

Thus, when checking position data stored in the indexes during the processes described herein, it may be said generally that the system may determine whether the position data for each k-mer meets predefined position criteria, which may vary depending on the application. In some embodiments, meeting predefined position criteria may require, as described above, that the position data indicates a specific absolute position. In some embodiments, meeting predefined position criteria may require, as described above, that the position data indicates a predefined offset number of bases from one or more previously matched k-mers, such that the system may determine that the matching k-mer strings continue to form a continuous conserved portion.

In some embodiments, position criteria (e.g., whether conserved regions are required to be located in the same position of each nucleic acid sequence in the first subset) or the absence thereof may be set in accordance with one or more inputs from a user of the system, for example a user of a user interface of device 170.

In order to determine that portions of a region are located in every one (or in a sufficient minimum percentage) of the nucleic acid sequences and, optionally, in a corresponding position in each nucleic acid sequence, the system may first look up the data stored in the first index corresponding to the initial position of the first nucleic acid sequence. The system may check what element of the index is pointed to by that data (or what element of the first index points to it), and the system may look for all other data in the first index that is associated with that data. (In embodiments in which a combined index is used, the system may only look for data that is indicated as being associated with the first subset of sequences, rather than looking at all data in the index associated with that element.) If the system determines that the first index includes one or more data structures associating that element with each of the other nucleic acid sequences in the first subset (and, optionally, that all of those data structures have corresponding position data), then the system may determine that the initial sub-sequence of length k of the first nucleic acid sequence is also located in each of the other nucleic acid sequences in the first subset (and, optionally, that it is located at a same or corresponding position).

It should be noted that, in some embodiments, whether position data is “corresponding” for multiple different nucleic acid sequences may be defined with respect whether the position data in each nucleic acid sequence bears the same relationship to position data for other data structures corresponding to other sub-strings for the same element. Thus, if a system is searching for a second sub-string located 16 bases further along the nucleic acid sequence from the first matching sub-string, then position data indicating a position 16 bases further along the sequence (regardless of the absolute position in any given nucleic acid sequence) may be said to be corresponding, while position data indicating a position elsewhere in a nucleic acid sequence may be said to be not corresponding. In this way, only matching sub-strings that continue to combine toward establishing a continuous matching region of length l may be returned as matching, while those that are located at another location in a nucleic acid sequence and do not contribute to combining toward establishing a continuous matching region of length l may not be counted.

It should also be noted that, when searching for an initial matching sub-string before any other matching sub-strings have been established, the search for matching sub-strings in the other nucleic acid sequences may be completely independent, such that matching data corresponding to the same element for another nucleic acid sequence may be satisfactory to establish a match, regardless of the position data associated with the other nucleic acid sequences for that element.

If the system fails to meet either of the above conditions with respect to locating data in the first index linking the same element to each of the nucleic acid sequences at matching or corresponding positions of each nucleic acid sequence, then the system may determine that the sub-string corresponding to the current position of the first nucleic acid sequence is not conserved across all of the nucleic acid sequences. In some embodiments, this negative determination may be attributable to one or more SNPs located in the relevant portion of one or more of the nucleic acid sequences in the first subset.

In accordance with this negative determination, the system may advance to a position in the first nucleic acid sequence following a mismatched base, and may then iterate the above-described process to determine whether the portion of the sequence following the mismatched base meets the above-described criteria. In some embodiments, when the system identifies which base or bases in the first nucleic acid sequence are not matched by every other nucleic acid sequence in the first subset (or by a sufficient number or percentage thereof), then the system may advance to the position corresponding to the base immediately following a mismatched base, and may begin the process of checking for matching data starting at that position. In some other embodiments, when the system cannot or does not determine which of the specific bases in the first nucleic acid sequence is responsible for the sub-string of k bases being determined to not match every other nucleic acid sequence in the index, the system may simply advance by one base (rather than to a specific base) and may again begin the process of checking for matching data starting at that position. In some embodiments, the system may advance to a position beyond the end of the entire sub-string (e.g., advancing by 16 bases at a time).

If, instead, a positive determination is made regarding matching or corresponding data being stored in the first index for every one of the nucleic acid sequences, it may be established that a k-mer of the first nucleic acid sequence matches with a corresponding k-mer of each other nucleic acid sequence, and the system may accordingly determine that it is possible that the k-mer of length k is included in (e.g., is the beginning of) a continuous conserved region of length l. The system may thus continue to scan down the length of the first nucleic acid sequence to determine whether a conserved region of length l can in fact be established.

In accordance with a positive determination that corresponding data is found in the first index for each of the nucleic acid sequences with respect to the given position, the system may determine whether a conserved region of length l has been established. In instances in which k=l, for example, establishing one matching k-mer across all nucleic acid sequences in the first subset may satisfy the condition of establishing a conserved region of length l across all nucleic acid sequences in the first subset. However, in other instances where k<l, merely establishing one matching sub-string of length k may not establish an entire conserved region of length l. Therefore, if all continuously matching portions identified by the system up to and including the most recent matching portion do not establish a portion of length l, then the system may need to continue to scan along the first nucleic acid sequence in order to determine that the next portion or portions continue to match, until a conserved region of length l can be established.

Accordingly, if it is determined that a conserved region of length l has not yet been established, then the system may advance to the first position in the first nucleic acid sequence following the end of the confirmed matching string. Because it has been established that matching data for the given position of the first nucleic acid sequence is located in the first index for all sequences in the first index, then it may be determined that the k-mer of the first nucleic acid sequence corresponding to the given position is also located in each of the other nucleic acid sequences in the first subset. Therefore, the system may shift down the first nucleic acid sequence by k bases in order to check whether the k-mer immediately following the established matching k-mer in the first nucleic acid sequence can also be established to match the next k bases in each of the other nucleic acid sequences in the first subset. In this manner, rather than exhaustively checking every base one at a time, the first index may allow for potentially conserved regions of length l to be established on a k-mer by k-mer basis, which may significantly reduce computational requirements and processing times.

After advancing to a position in the first nucleic acid sequence immediately following the most recently established matching k-mer or k-mers, the technique may then iterate until one or more conserved regions of length l are established.

If it is positively determined that all continuously matching portions identified by the system up to and including the most recent matching portion do together establish a continuously matching portion of length l across all of the nucleic acid sequences in the subset, then the technique may proceed to block 310.

At block 310, in some embodiments, the system may, for each conserved region identified, using the second index, determine whether the conserved region is a signature region that is not identical to any region in any nucleic acid sequence of the second subset. In some embodiments, in addition to or alternatively determining whether a conserved region appears in none of the nucleic acid sequences of the second subset, the system may identify those conserved regions that appear in fewer than or equal to a maximum threshold percentage of nucleic acid sequences of the second subset. Reference in the specification may be made to determining that a region is signature as compared to all nucleic acid sequences in a subset, but it is to be understood that the same techniques may be applied to ensure that a region is conserved for at least a threshold percentage of nucleic acid sequences in a subset. (In some embodiments, the threshold maximum number of sequences may be configurable by a user input, for example by a user input received from a user of user device 170.) In the example of system 100 of FIG. 1, the processing steps of block 310 may be performed by kit configuration engine 150.

In some embodiments, for each matching region of length l identified, the system may determine that the region is a conserved region and that is it potentially a conserved-signature region that can be loaded onto the kit for use in sample identification protocols. The system may thus undertake to determine whether the identified conserved region (conserved with respect to the first subset) is in fact also a signature region (signature with respect to the second subset). The system may determine, for example, that the conserved region is likely to be present in a target organism or organisms that the sample identification protocol will search for. However, the system may not yet be aware of whether the conserved region is also likely to be present in sequences associated with other organisms that the system is not targeting, which would therefore create the risk of false-positive results in the sample identification protocol. Determining whether a region is signature against the second subset of sequences therefore ensures that the conserved region will be adequately discriminatory against nucleic acid sequences in the second subset. Thus, determining that the region is conserved may ensure that targeting the region will not result in false-negative failure to identify sequences associated with the target organism(s), but it may not ensure that targeting the region will not result in false-positive selection for sequences not associated with the target organism(s). Accordingly, the system may proceed as described herein to determine if the conserved region is adequately discriminatory.

As described below, the process of determining whether a conserved sequence is signature may be performed based on the second index, which may have been created based on the second subset of nucleic acid sequences (e.g., using k-mers taken therefrom) according to the techniques described above.

In some embodiments, the system may determine, for each conserved region identified, whether the region is identical to any region in any nucleic acid sequence outside the subset. As described below, this determination may be made by comparing data stored in the first index to data stored in the second index in order to quickly and efficiently determine whether or not the identified conserved region is unique against all nucleic acid sequences in the second subset.

In some embodiments, the system may determine whether, for the initial position in the conserved region, data stored in the first index corresponds to the same element as data stored in the second index. Thus, the system may look up, in the first index, the data indicating the initial position of the conserved region. The system may note the element of the first index to which the data for the initial position of the conserved region points, and the system may then check in the second index for any data stored that points to (or is pointed from) the corresponding (e.g., same) element. (In embodiments where a single combined index is used, the system may determine whether data stored in association with the first subset corresponds to data stored in association with the second subset and in association with the same element of the combined index.)

If no such data is found in the second index (or if data found in the second index indicates that the sequence is found in a sufficiently low number or percentage of sequences in the second subset), then the system may determine that the data stored in the first index for the initial position in the conserved region does not correspond to the same element as any data stored in the second index for any of the nucleic acid sequences (and indeed does not correspond to the same element as any of the data stored in the second index at all). In these cases, the system may determine that the conserved region is a conserved-signature region amenable to use in a sample identification protocol to target organism(s) in the first subset and to distinguish organism(s) in the second subset.

Thus, the system may determine, for a conserved region determined to not be identical to any region in any nucleic acid sequence in the second subset, that the conserved region is a discriminatory region amenable for use in a sample identification protocol to target organism(s) in the first subset and to distinguish organism(s) in the second subset. As discussed above, it may be determined that no data in the second index corresponds to the same element as the element corresponding to the first portion of the conserved region, thus indicating that the initial sub-string of length k of the conserved region is not found anywhere in any of the nucleic acid sequences seeded into the second index. That is, the initial sub-string of length k of the conserved region may be determined to be unique against all of the nucleic acid sequences in the second index, therefore establishing that the entire conserved region is necessarily unique against all strings of length l in the nucleic acid sequences in the second index (due at least, but not necessarily exclusively, to the unique sub-string of length k beginning at the initial position of the conserved region). Thus, the system may determine that the conserved region is both (a) conserved among all members of the first subset and (b) unique against all members of the second subset, therefore making the region a conserved-signature region potentially amenable for use in a sample identification protocol to target organism(s) in the first subset and to distinguish organism(s) in the second.

If, on the other hand, the system determines that, for the initial position in the conserved region, data stored in the first index does corresponds to the same element as data stored in the second index for any (or for a sufficiently high percentage or number of) nucleic acid sequences in the second subset, the system may continue to analyze the conserved region because the initial position does not establish that the conserved region is also a signature region.

While the absence of any data in the second index corresponding to the matching index associated with the initial position of the conserved region may indicate that no sub-string matching the initial sub-string in the conserved region exists anywhere in any nucleic acid sequence outside the subset, the presence of any such data may indicate the opposite. That is, in some embodiments, the presence of data seeded into the second index at the element corresponding to the relevant element from the first index (e.g., the matching or identical element), may indicate that at least one of the nucleic acid sequences in the second subset contains an identical sub-string to the sub-string defining the first k bases of the conserved region. In these cases, the system may thus determine that the data stored in the first index for the initial position in the conserved region does correspond to the same element as data stored in the second index for one or more of the nucleic acid sequences in the second subset.

In some embodiments, when the second index contains additional data corresponding to additional nucleic acid sequences against which the system is not checking the conserved region, then the system may perform an additional check to determine whether any of the data pointing to or being pointed from the relevant element in the second index corresponds to a relevant nucleic acid sequence against which the system is comparing the conserved region.

In these cases, because it is determined that the sub-string defining the first k bases on the conserved region matches a sub-string of k bases somewhere in one of the nucleic acid sequences represented in the second index, the system may continue to check the nucleic acid sequences represented in the second index that show a matching sub-string of length k, to determine whether an any portion of the conserved region of length l can be established to be signature against the second subset. In response to determining that the sub-string defining the first k bases on the conserved region matches a sub-string of k bases somewhere in one of the nucleic acid sequences represented in the second index, the technique may proceed down the conserved region as described below.

In some embodiments, the system may determine whether the end of the conserved region has been reached. The system may determine whether the portion(s) of the conserved region that have been established to match a portion of one or more of the nucleic acid sequences in the second index account for the entirety of the conserved region.

For example, in embodiments where k=l, this condition may be satisfied after establishing that any one sub-string (e.g., the initial element checked) in the conserved region matches a sub-string in one of the nucleic acid sequences in the second element. If this is the case, then the system may determine that the entire conserved region 1 matches at least one continuous portion in one of the sub-strings outside the subset and represented by the second index, and may therefore determine that the conserved region is a not a conserved-signature region. Thus, the system may determine, for a conserved region determined to be identical to at least one continuous region in at least one other nucleic acid sequence outside the subset, that the conserved region is not a discriminatory region that would be usable for targeting the target organism(s) and for distinguishing the organism(s) in the second subset. The system may determine that, while the conserved region is consistent among all members of the first subset, it is not unique against all members of the second subset. Therefore, searching for the region in sample sequences could select for nucleic acid sequences in the second subset, therefore generating false positive search results. In these instances, the system may discard the conserved sequence, or the system may store an indication (e.g., for future reference) that the sequence is not a conserved-signature sequence for the first subset and the second subset (e.g., such that the full conserved-signature identification process can optionally be circumvented in the future if the same first and second subset are selected for configuration of a kit.)

It may instead be determined that the end of the conserved region has not been reached in the comparison of the conserved region against the nucleic acid sequences in the second index. For example, when k<l, mere determination that the first k bases of the conserved region appear in at least one nucleic acid sequence represented by the second index may be insufficient to determine that the entire conserved region 1 appears in any one of the nucleic acid sequences represented by the second index. Accordingly, the system may proceed down the conserved region to check the next portion, and the portion after that, and so on, to determine whether any of the nucleic acid sequences outside the subset indeed include a string that matches the entirety of the conserved region.

Thus, the technique may advance to the position in the conserved region following the end of the matching conserved region sub-string. The matching conserved region sub-string may refer to the most recent and/or furthest advanced sub-string that has been determined by the system to match one or more sub-strings in one of the nucleic acid sequences represented by the second index. Thus, following an initial determination that the k-mer beginning at the initial position of the conserved region also appears in one or more of the nucleic acid sequences of the second subset, the system may advance to the k-mer beginning at the (k+l) position of the conserved region.

In some embodiments, the system may determine, for the position in the conserved region following the end of the matching conserved region sub-string, whether the data stored in the first index corresponds to the same element as data stored in the second index for any nucleic acid sequence established to have a matching sub-string, for the position in the respective nucleic acid sequence following the end of its matching sub-string. That is, after advancing to the position in the conserved region following the end of the most recent sub-string in the conserved region determined to match one or more sub-strings in a nucleic acid sequence of the second index, the system may check where the data corresponding to that position for the conserved region has been seeded into the first index. To do this, the system may look up the data corresponding to the new position in the conserved region, and may check which element in the first index is pointed to. The system may then turn to the second index, and may look up all data that points to (or is pointed to by) the corresponding (e.g., matching) element in the second index. Any data that has been seeded into the second index to correspond to the matching element in the second index may indicate that at least one nucleic acid sequence in the second index contains a sub-string that matches the sub-string of the conserved region that begins at the new position.

However, unlike the above-described analysis of whether an initial portion of a conserved region is represented anywhere in the second index, merely establishing that a matching sub-string (e.g., a matching k-mer) exists anywhere in any of the nucleic acid sequences represented by the second index may not be dispositive of whether any region potentially matching the conserved region exists. Here, since the system has already determined which nucleic acid sequences of the second subset have sub-strings matching part of the conserved region, the system may only be interested in further analysis of the same nucleic acid sequences of the second subset that are already indicated as potentially matching the entire conserved region. Furthermore, because a continuous matching region of all l bases in the same order as the conserved region may be required to establish that the conserved region is not unique against the second subset, the system must also establish that any matching sub-string in the relevant nucleic acid sequence of the second subset is located at the position following the end of the sub-string that was most recently established to match a sub-string of the conserved region. Put simply, the system may seek to check whether any of the nucleic acid sequences of the second subset established as matching an initial portion of the conserved region continue to match the next portions of the conserved region in the next portions of that nucleic acid sequence.

In order to do this, the system may read any data found in the second index associated with the matching element looked up in the first index, and may check whether any of that data (a) corresponds to a nucleic acid sequence that was previously established to match all previously checked portions of the conserved region, and (b) corresponds to the position in the previously matching nucleic acid sequence following its previously matched portion. (In some embodiments, satisfying criteria (b) may be referred to as satisfying position criteria. Generally speaking, satisfying position criteria while checking for uniqueness against the second index may require that a potentially matching portion is determined to appear in the correct spatial/positional relation to all other matching portions of the region. When checking an initial sub-string of a conserved portion against the second index, all matching k-mers indicated in the second index may be determined to satisfy position criteria; thereafter, additional k-mers may be required to be located adjacent to and/or immediately after previously-matched k-mers in the nucleic acid sequence of the second index in order to satisfy position criteria.) In some embodiments, the system may do this by checking whether any data structure stored in association with the relevant index has the same identification metadata as a nucleic acid sequence previously matching all checked portions of the conserved region, and whether the position data associated with that data structure indicates a position immediately following all previously matching portions in the relevant nucleic acid sequence. Thus, if it is established that both conditions are met (e.g., that a data structure in the second index associates the relevant element with a previously matching nucleic acid sequence at the immediate next portion of the nucleic acid sequence), then the system may determine that the conserved region continues to match at least one portion of at least one of the nucleic acid sequences in the second subset.

In accordance with the determination above (e.g., that the conserved region continues to match at least one portion of at least one of the nucleic acid sequences represented by the second index), the system may again and determine whether the entire length l of the conserved region has been checked and accounted for by any of the one or more matching nucleic acid sequences in the second index. If, accounting for the sum of all of the contiguous matched sub-strings in any one given nucleic acid sequence, the entire conserved region has been matched (e.g., the end of the conserved region has been reached), then the system may determine that the conserved region is not a signature region, as described above. If, accounting for the sum of all of the contiguous matched sub-strings in any one given nucleic acid sequence, the entire conserved region has not yet been matched (e.g., the end of the conserved region has not yet been reached), then the technique may continue advancing further down the conserved region and continue to iterate the process described herein to check whether the potentially matching regions of the nucleic acid sequences continue to match the conserved region.

However, if during any iteration of the process for checking if a portion of a conserved region matches data in the second index, it is determined that any of the conditions explained above (e.g., matching criteria and position criteria) are not met for any remaining nucleic acid sequences in the second subset, then the technique may determine that the conserved region is in fact a conserved-signature region that is potentially amenable for use as a conserved-signature sequence to target one or more organisms in the first subset and to distinguish one or more organisms in the second subset in a sample identification protocol. For example, if, for any element in the first index corresponding to the position in the conserved region currently being checked, the matching element in the second index does not contain a data structure matching both the identity of a previously matched nucleic acid sequence and the next position in that nucleic acid sequence following the insofar matching portion, then the system may immediately determine that the conserved region does not match the nucleic acid sequence at the portion being checked, and that the entire conserved region is therefore not identical to any portion of the nucleic acid sequence. The system may thus determine, if a non-matching element is established for every nucleic acid sequence in the second index (or for a sufficient number or percentage thereof), that the conserved region is indeed a conserved-signature region amenable for use in configuring the kit for performing sample identification based on the conserved-signature region.

In some embodiments, when a system is locating conserved regions of minimum length l, the system may discover a conserved region of length l but may not stop scanning down the first nucleic acid sequence, and may not jump to the end of the conserved region to begin scanning again from that location. Instead, by continuing to scan down the nucleic acid sequence on a base by base or k-mer by k-mer basis, until a non-matching sub-string and/or non-matching base is encountered, the system may establish a conserved region of a length greater than l. In this way, the system may establish a plurality of conserved regions of length l, each partially overlapping and shifted by just one base from one another, each of which may be tested to determine whether it is unique against nucleic acid sequences in the second subset. Thus, if the first conserved region of length l is determined not to be unique against all nucleic acid sequences outside the subset, then the system may be able to shift down by one base at a time and check whether any of those conserved regions of length l are unique.

The description above pertaining to blocks 304-310 refers to lengths l and k for creation of indexes and use of indexes to identify conserved-signature regions. It should be noted that different lengths of l and k may be used in different use cases.

In some embodiments, different lengths of k (e.g., a length of k-mers for creation of the indexes and for k-mer-by-k-mer analysis of the indexes) may be selected in consideration processing resources and/or storage resources available to one or more systems performing the operations to create the indexes and analyze the indexes to identify conserved-signature regions. In some embodiments, the system may automatically select a length k based on one or more predefined default settings, based on data indicating available storage resources, available processing resources, or available processing times—each of which may be separately considered for the index creation processes and for the conserved-signature sequence identification index-analysis processes. In some embodiments, the system may select a length k based on a user input, such as a user input provided via a graphical user interface of system 170.

In some embodiments, different lengths of l (e.g., a minimum length of a sequence that can be identified as a conserved sequence and/or a conserved-signature sequence) of may be selected by a system and/or may be manually settable and adjustable by a user. In some embodiments, the system may automatically select a length l based on one or more predefined default settings.

In some embodiments, a length l may be selected in consideration processing resources, storage resources, and/or hardware to be used by the portable kit to performing the operations to sequence sample sequences and to identify said sample sequences based on the conserved-signature regions. In some embodiments, the system may automatically select a length l based on data indicating available storage resources, available processing resources, or hardware to be used by the portable kit. For example, if the portable kit is to use a sequencing system that will generate reads of a certain length, then the system may select a length l based in part on that length, for example such that the system does not seek to identify conserved-signature sequences that are longer than reads that will be sequenced by the kit. In some embodiments, the system may select a length l based on a user input, such as a user input provided via a graphical user interface of system 170. The user input for selecting a length l may comprise an explicit indication of a length l (which may in some embodiments indicate a minimum length, a maximum length, and/or an exact length for conserved-signature sequences). Alternatively or additionally, the input for selecting a length l may comprise an indication of information about the kit to be configured (e.g., an identification, such as an identification number, of the kit and/or of one or more components of the kit), and the system may automatically select (or suggest for user confirmation) a length l based on the information provided about the kit to be configured.

In some embodiments, longer lengths l may yield fewer sub-strings that are conserved (e.g., common, identical, matching) across all members of the first subset, while they may be more likely to yield conserved sub-strings that are unique as compared to all known nucleic acid sequences outside the first subset (e.g., sequences in the second subset). In some embodiments, a specific length l, or a length l within a certain range, may be desirable for sample identification; for example, lengths l of longer than 25, 50, 75, 100, 125, 150, or 200 base pairs may be desirable, while lengths l of shorter than 250, 225, 200, 175, 150, or 125 base pairs may be desirable.

Techniques for identifying conserved-signature sequences that may be used in or with the systems, methods, and/or techniques described herein are described in U.S. patent application Ser. No. 15/977,659, titled “Primer Design Using Indexed Genomic Information,” which application is hereby incorporated by reference in its entirety.

Following identification of one or more conserved-signature regions, for example in accordance with the above techniques, the system may proceed to block 312.

At block 312, in some embodiments, the system may generate data representing the one or more identified conserved-signature regions for storage on a portable computer storage medium of a portable kit for nucleic acid sequencing and sample identification.

A sequence that is identified as a conserved-signature sequence may be transmitted to an electronic storage medium of the portable sequencing and sample identification kit for storage thereon, thereby configuring the kit for efficient and effective identification of target organisms in a specifically-contemplated deployment situation. As described above with reference to FIG. 1, identified conserved-signature sequences may be stored on a portable kit in a manner that explicitly represents the entire sequence identity of the conserved-signature sequence, and/or they may be represented on a portable kit by a probabilistic data structure that represents conserved-signature sequences as members of a set. By configuring the kit using the identified conserved-signature sequences in this manner, the kit may be able to effectively and efficiently identify nucleic acid sequences associated with the target organism without need to have access to the entirety of an extensive nucleic acid database or library. The configured kit 101 may then be used for sequence identification, for example as described above with reference to FIG. 1, in situations in which network communication is not available or is ineffective.

In the example of system 100 of FIG. 1, kit configuration engine may transmit the identified conserved-signature sequences, or data representing said identified conserved-signature sequences (e.g., one or more probabilistic data structures), to kit 101 for storage on storage medium 140.

In some embodiments, in additional or alternatively to performing one or more of the techniques described herein to identify a custom set of conserved-signature sequences based on a first subset and a second subset of selected nucleic acid sequences, the system may configure a portable kit for deployment by selecting one or more predefined (rather than custom-made) sets of conserved-signature sequences. For example, in situations in which a user does not have specific knowledge regarding non-targeted organisms that are likely to be present in an environment of the targeted organism, the user may simply wish to target the target organisms using conserved-signature sequences that distinguish all other known nucleic acid sequences, or the largest reasonably possible number of other known nucleic acid sequences. In this case, the system may perform the above process for identifying conserved-signature sequences, designating all known non-targeted sequences as being included in the second set. In some embodiments, rather than performing the above process for identifying conserved-signature sequences, the system may access a predetermined set of conserved-signature sequences that have already been identified. For example, the system may have access a pre-saved selection of conserved-signature sequences, such that a user may select from among the pre-saved selection to load the selected conserved-signature sequences onto the kit. In some embodiments, a user may make a selection, e.g., via a graphical user interface of user device 170, as to whether the user wishes to select from predefined conserved-signature sequence sets or if the user wishes to generate custom conserved-signature sequences. If the user wishes to select from predefined conserved-signature sequence sets, the user may then make a selection via the graphical user interface.

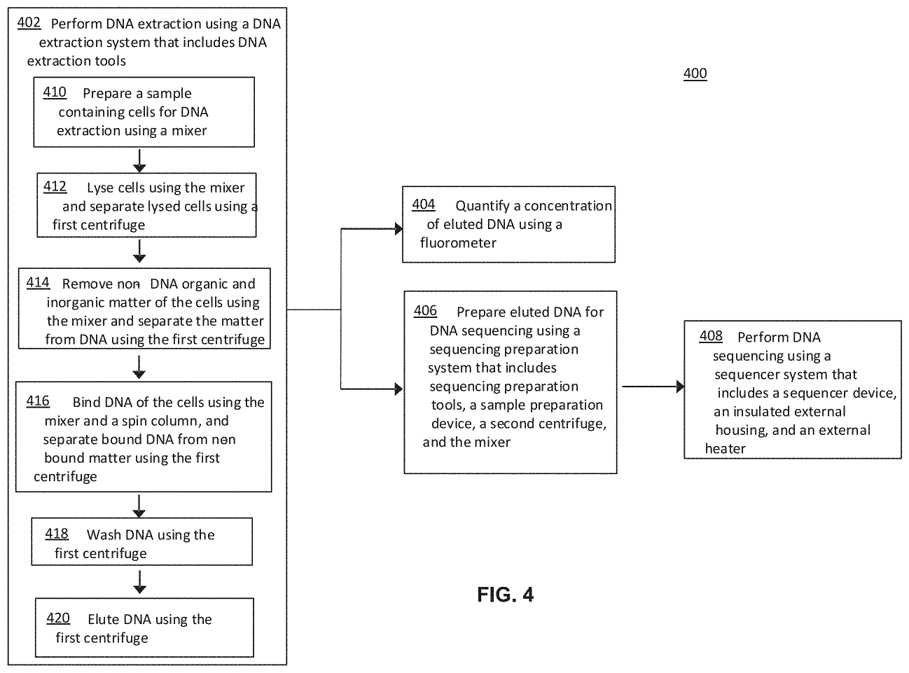

FIG. 4 shows a method 400 for performing nucleic acid sequencing using a portable nucleic acid sequencing kit, such as kit 101, according to some embodiments. In step 402, a sample containing DNA may be processed by the DNA extraction system (such as extraction system 104). The DNA extraction system may be configured to output eluted DNA. In step 404, the eluted DNA may be quantified by a fluorometer. In step 406, the eluted DNA may be received by the DNA sequencing preparation system (such as preparation system 106). The preparation system may be configured to output DNA prepared for sequencing. In step 408, the sequencing system (such as 110) may be configured to output sequencing data of the extracted DNA.

In some embodiments, step 402 may include a preparation step 410, a lyse step 412, a removal step 414, a binding step 416, a wash step 418, and an elution step 420. In some embodiments, preparation step 410 includes preparing a sample containing cells for DNA extraction using a mixer (such as mixer 122). In some embodiments, the lysis step 412, the removal step 414, and the binding step 416 may include utilization of a mixer and a centrifuge (such as the mixer 122 and the first centrifuge 118). In some embodiments, the wash step 418 and the elution step 420 may include utilization of a first centrifuge (such as the first centrifuge 118).

In some embodiments, step 406 may include utilization of sequencing preparation tools, a sample preparation device, a second centrifuge, and a mixer (such as preparation tools 124, separation device 126, second centrifuge 120, and mixer 122).

In some embodiments, step 408 may include utilization of a sequencer device (such as sequencer device 128) and a heater (such as heater 130) positioned within an insulated casing (such as insulated housing 132).

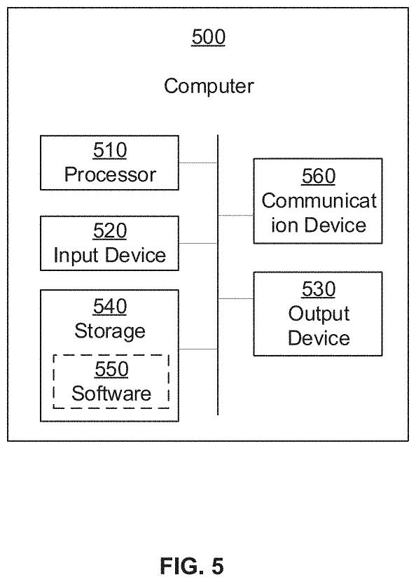

FIG. 5 depicts an example of a computer system that may be implemented as part of any one or more of the systems, devices, or subcomponents described herein, and that may be configured to perform all or part of any one or more of the methods or techniques described herein, in accordance with some embodiments. As shown in FIG. 5, system 500 can be any suitable type of microprocessor-based device, such as a personal computer, workstation, server, handheld computing device, such as a phone or tablet, or distributed computing system (e.g., cloud computing system). The system can include, for example, one or more of processor 502, communication device 504, input device 506, output device 508, storage 510, and/or software 512 stored on storage 510 and executable by processor 502. The components of the computer can be connected in any suitable manner, such as via one or more physical buses or wirelessly.

In some embodiments, system 500 may include server-side computing components as well as client-side computing components. The specific elements shown in FIG. 5 may, in some embodiments, be included in a server-side computer and/or may, in some embodiments, be included in a client-side computer. In some embodiments, system 500 may include server-side components and client-side components that are in communication with one another via one or more instances of communication device 504, which may, for example, enable communication of server-side components and client-side components over a network connection.

In some embodiments, some or all components of system 500 may be part of a distributed computing system (e.g., a cloud computing system). In some embodiments of the techniques disclosed herein, for example, storage 510 may be storage provisioned by a cloud computing system, such that a user may send instructions to the cloud computing system over one or more network connections, and the cloud computing system may execute the instructions in order to leverage the cloud computing components in accordance with the instructions. In some embodiments, cloud computing systems may be configured to be capable of executing the same or similar program code in the same programming languages as other systems (e.g., servers, personal computers, laptops, etc.) as discussed herein.

Processor 502 may be any suitable type of computer processor capable of communicating with the other components of system 500 in order to execute computer-readable instructions and to cause system 500 to carry out actions in accordance with the instructions. For example, processor 500 may access a computer program (e.g., software 512) that may be stored on storage 510 and execute the program to cause the system to perform various actions in accordance with the program. In some embodiments, a computer program or other instructions executed by processor 502 may be stored on any transitory or non-transitory computer-readable storage medium readable by processor 502.

Communication device 504 may include any suitable device capable of transmitting and receiving signals over a network, such as a network interface chip or card. System 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Input device 506 may be any suitable device that provides input, such as a touch screen or monitor, keyboard, mouse, button or key or other actuatable input mechanism, microphone, and/or voice-recognition device, gyroscope, camera, or IR sensor. Output device 508 may be any suitable device that provides output, such as a touchscreen, monitor, printer, disk drive, light, speaker, or haptic output device.

Storage 510 can be any suitable device that provides storage, such as an electrical, magnetic or optical memory including a RAM, cache, hard drive, CD-ROM drive, tape drive, or removable storage disk.

Software 512, which may be stored in storage 510 and executed by processor 502, may include, for example, the programming that embodies the functionality of the methods, techniques, and other aspects of the present disclosure (e.g., as embodied in the computers, servers, devices, components, and/or subcomponents as described above). In some embodiments, software 512 may include a combination of servers such as application servers and database servers.

Software 512 can also be stored and/or transported within any computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 510, that can contain or store programming for use by or in connection with an instruction execution system, apparatus, or device.

Software 512 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

System 500 can implement any one or more operating systems suitable for operating on the network. Software 512 can be written in any one or more suitable programming languages, such as C, C++, Java, or Python. In various embodiments, application software embodying the functionality of the present disclosure can be deployed in different configurations, such as in a client/server arrangement or through a Web browser as a Web-based application or Web service, for example.

Although the description herein uses terms first, second, etc., to describe various elements, these elements should not be limited by the terms. These terms are only used to distinguish one element from another.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components but do not preclude the presence or addition of one or more other features, integers, steps, operations, elements, components, and/or groups thereof.

The term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

This application discloses several numerical ranges in the text and figures. The numerical ranges disclosed inherently support any range or value within the disclosed numerical ranges, including the endpoints, even though a precise range limitation is not stated verbatim in the specification, because this disclosure can be practiced throughout the disclosed numerical ranges.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A portable kit for nucleic acid sequencing and sample identification, comprising:
a DNA extraction system configured to perform a DNA extraction protocol to extract DNA from a sample;
a DNA sequencing preparation system configured to perform a DNA sequencing preparation protocol on the extracted DNA;
a sequencer system configured to generate sample nucleic acid sequence data for the extracted DNA;
memory storing reference nucleic acid data representing a plurality of reference nucleic acid sequences;
one or more processors configured to compare the sample nucleic acid sequence data to the reference nucleic acid data to generate output data indicating one or more organisms with which the sample is determined to correspond; and
a portable enclosure configured to house the DNA extraction system, the sequencer preparation system, the sequencer system, the memory, and the one or more processors.

2. The portable kit of claim 1, wherein the reference nucleic acid data represents one or more target regions identified by:
determining, by a first index comprising data representing a first set of nucleic acid sequences, that the target region is a conserved region appearing in every nucleic acid sequence in the first set; and
confirming, by the second index comprising data representing a second set of nucleic acid sequences, that the conserved region appears in none of the nucleic acid sequences in the second set.

3. The portable kit of claim 1, wherein the reference nucleic acid data comprises sequence data and associated metadata, wherein the associated metadata indicates an organism associated with the target region.

4. The portable kit of claim 3, wherein the associated metadata indicates a type of organism associated with the target region.

5. The portable kit of claim 1, wherein the reference nucleic acid data comprises a probabilistic data structure that represents one or more of the plurality of reference nucleic acid sequences as members of a set.

6. The portable kit of claim 5, wherein comparing the sample nucleic acid sequence data to the reference nucleic acid data comprises querying the probabilistic data structure by data representing the sample nucleic acid sequence to responsively generate data indicating whether the sample nucleic acid sequence is a member of the set.

7. The portable kit of claim 5, wherein:
the probabilistic data structure is stored as part of a multi-level data structure comprising a plurality of hierarchically-interrelated probabilistic data structures;
probabilistic data structures in a first level of the multi-level data structure represent respective sets of the plurality of reference nucleic acid sequences; and
probabilistic data structures in a second level of the multi-level data structure represent respective subsets of the sets of the plurality of reference nucleic acid sequences.

8. The portable kit of claim 7, wherein:
data structures in the first level of the multi-level data structure represent respective sets of the plurality of reference nucleic acid sequences that are associated with a respective type of organism; and
data structures in the second level of the multi-level data structure represent respective sets of the plurality of reference nucleic acid sequences that are associated with a respective organism.

9. The portable kit of claim 1, wherein the output data comprises ranking data indicating a respective match strength for each of the one or more organisms with which the sample is determined to correspond.

10. The portable kit of claim 9, wherein the respective match strength is determined based on a number of sequences in the reference nucleic acid data to which the sample nucleic acid sequence data is determined to correspond.

11. The portable kit of claim 1, wherein the memory is configured to be selectively loaded with different sets of reference nucleic acid data representing different pluralities of reference nucleic acid sequences.

12. The portable kit of claim 11, wherein one of the different pluralities of reference nucleic acid sequences corresponds to a predefined type of organism, including one or more of the following: biological warfare agents, food pathogens, viruses, bacteria, fungi, mammalian, and harmful agents.

13. The portable kit of claim 1, comprising a first centrifuge and a portable power supply, wherein:
the first centrifuge is configured to ramp to a target speed at a first ramp rate when drawing power from the portable power supply, and
the first centrifuge is configured to ramp to the target speed at a second ramp rate, faster than the first ramp rate, when drawing power from a source of line power.

14. The portable kit of claim 13, wherein ramping at the first ramp rate comprises increasing from an initial speed to the target speed in predetermined increments.

15. The portable kit of claim 1, wherein the sequencer system comprises a sequencer device, a heating device positioned external to the sequencing device, and an insulated casing that houses the sequencing device and the heating device.

16. A system for configuring a kit for nucleic acid sequencing and sample identification, comprising:
a first set of one or more processors configured to:
receive genomic data representing a first set of one or more nucleic acid sequences;
create and store data in a first index representing a first set of nucleic acid sequences;
receive genomic data representing a second set of one or more nucleic acid sequences;
create and store data in a second index representing the second set of nucleic acid sequences; and
identify a target region to serve as a nucleic acid reference sequence that corresponds to one or more of the nucleic acid sequences in the first set and that discriminates against one or more of the nucleic acid sequences in the second set, wherein the identifying comprises:
identifying, by the first index, the target region as a conserved region appearing in every nucleic acid sequence in the first set; and
confirming, by the second index, that the conserved region appears in none of the nucleic acid sequences in the second set; and
a portable kit for nucleic acid sequencing and sample identification, the portable kit comprising a second set of one or more processors and memory;
wherein the first set of one or more processors are configured to cause transmission of data representing the target region to the portable kit for storage on the memory;
wherein the second set of one or more processors are configured to compare the target region to a sample nucleic acid sequence to determine whether the target region matches the sample nucleic acid sequence.

17. The system of claim 16, wherein:
receiving the genomic data representing the first set comprises receiving a first user input indicating the one or more nucleic acid sequences; and
receiving the genomic data representing the first set comprises receiving a second user input indicating the one or more nucleic acid sequences.

18. The system of claim 17, wherein the first user input comprises selection of an organism from a menu.

19. The system of claim 17, wherein the first user input comprises selection of a type of organisms from a menu.

20. The system of claim 17, wherein the second user input comprises selection of an organism from a menu.

21. The system of claim 17, wherein the second user input comprises selection of a type of organisms from a menu.

22. The system of claim 16, wherein the first set of one or more processors configured to receive a length input indicating a base length for the target region to be identified.

23. The system of, claim 16 wherein:
the first set of one or more processors is configured to receive an input indicating an index base-length to be used in creation of the first index; and
creating and storing data in a first index representing the first set of nucleic acid sequences comprises representing the first set of nucleic acid sequences using subsequences having a length equal to the indicated index base-length.

24. The system of claim 23, wherein the input indicating the index base-length comprises one or more of the following:
a user input explicitly specifying a number of bases;
data characterizing processing resources of the first set of one or more processors; and
data characterizing storage resources available for storage of the first index.

25. The system of claim 16, wherein:
the first set of one or more processors is configured to receive an input indicating a target region base-length criteria to be used in identification of the target region; and
identifying the target region comprises ensuring that the identified target region has a length that complies with the indicated target region base-length criteria.

26. The system of claim 25, wherein the input indicating the target region base-length criteria comprises one or more of the following:
a user input explicitly specifying a number of bases;
data characterizing processing resources of the first set of one or more processors;
data characterizing storage resources available for storage of the first index;
data characterizing processing resources of the second set of one or more processors; and
data characterizing storage resources available on the memory for storage of the conserved-signature sequences on the portable kit.

27. The system of, claim 25 wherein the input indicating the target region base-length criteria comprises data characterizing a base length of sample nucleic acid sequences generated by a sequencing system of the portable kit.

28. The system of claim 16, wherein the data representing the target region comprises sequence data and associated metadata, wherein the associated metadata indicates an organism associated with the target region.

29. The system of claim 16, wherein the data representing the target region comprises sequence data and associated metadata, wherein the associated metadata indicates a type of organism associated with the target region.

30. The system of claim 16, wherein the data representing the target region comprises a probabilistic data structure that represents the target region as a member of a set.

31. The system of claim 30, wherein comparing the target region to a sample nucleic acid sequence comprises querying the probabilistic data structure by data representing the sample nucleic acid sequence to responsively generate data indicating whether the sample nucleic acid sequence is a member of the set.

32. The system of, claim 30 wherein the first set of one or more processors are configured to:
receive a false-positivity probability input; and of the probabilistic data structure; and
set a false-positivity rate for the probabilistic data structure in accordance with the false-positivity probability input.

33. The system of, claim 30 wherein:
the probabilistic data structure is stored as part of a multi-level data structure comprising a plurality of hierarchically-interrelated probabilistic data structures;
probabilistic data structures in a first level of the multi-level data structure represent respective sets of the plurality of reference nucleic acid sequences; and
probabilistic data structures in a second level of the multi-level data structure represent respective subsets of the sets of the plurality of reference nucleic acid sequences.

34. The system of claim 33, wherein:
data structures in the first level of the multi-level data structure represent respective sets of the plurality of reference nucleic acid sequences that are associated with a respective type of organism; and
data structures in the second level of the multi-level data structure represent respective sets of the plurality of reference nucleic acid sequences that are associated with a respective organism.

35. The system of claim 33, wherein the first set of one or more processors are configured to:
receive a multi-level data-structure arrangement input; and
define an arrangement of the multi-level data structure in accordance with the multi-level data-structure arrangement input.

36. The system of claim 16, wherein:
the data representing the target region comprises an index representing the target;
the index comprises a plurality of data structures representing respective sub-string of the target region; and
the respective data structures are stored in the index and indicate an identity of the target region, a permutation of bases forming the sub-string of the target region, and a position of the sub-string in the target region.

37. The system of claim 36, wherein comparing the target region to a sample nucleic acid sequence comprises determining whether the index stores a data structure associated with a sub-string of the sample nucleic acid sequence.

38. The system of claim 16, wherein creating and storing data in the first index comprises:
for each of the nucleic acid sequences in the first set, dividing the nucleic acid sequence into a plurality of sub-strings;
for each of the plurality of sub-strings, storing a data structure in the first index, wherein:
the data structure indicates an identity of the nucleic acid sequence, a permutation of bases forming the sub-string, and a position of the sub-string in the nucleic acid sequence.

39. A non-transitory computer-readable storage medium storing instructions for configuring a kit for nucleic acid sequencing and sample identification, wherein the instructions are configured to be executed by a system comprising one or more processors to cause the system to:
receive genomic data representing a first set of one or more nucleic acid sequences;
create and store data in a first index representing a first set of nucleic acid sequences;
receive genomic data representing a second set of one or more nucleic acid sequences;
create and store data in a second index representing the second set of nucleic acid sequences;
identify a target region to serve as a nucleic acid reference sequence that corresponds to one or more of the nucleic acid sequences in the first set and that discriminates against one or more of the nucleic acid sequences in the second set, wherein the identifying comprises:
identifying, by the first index, the target region as a conserved region appearing in every nucleic acid sequence in the first set; and
confirming, by the second index, that the conserved region appears in none of the nucleic acid sequences in the second set; and
transmit data representing the target region to a portable kit for storage on memory of the portable kit, wherein the portable kit is configured to compare the target region to a sample nucleic acid sequence to determine whether the target region matches the sample nucleic acid sequence.

40. A method for configuring a kit for nucleic acid sequencing and sample identification, the method performed at a system comprising one or more processors, the method comprising:
receiving genomic data representing a first set of one or more nucleic acid sequences;
creating and storing data in a first index representing a first set of nucleic acid sequences;
receiving genomic data representing a second set of one or more nucleic acid sequences;
creating and storing data in a second index representing the second set of nucleic acid sequences;
identifying a target region to serve as a nucleic acid reference sequence that corresponds to one or more of the nucleic acid sequences in the first set and that discriminates against one or more of the nucleic acid sequences in the second set, wherein the identifying comprises:
identifying, by the first index, the target region as a conserved region appearing in every nucleic acid sequence in the first set; and
confirming, by the second index, that the conserved region appears in none of the nucleic acid sequences in the second set; and
transmitting data representing the target region to a portable kit for storage on memory of the portable kit, wherein the portable kit is configured to compare the target region to a sample nucleic acid sequence to determine whether the target region matches the sample nucleic acid sequence.

41. A system for configuring a kit for nucleic acid sequencing and sample identification, the system comprising one or more processors configured to:
receive genomic data representing a first set of one or more nucleic acid sequences;
create and store data in a first index representing a first set of nucleic acid sequences;
receive genomic data representing a second set of one or more nucleic acid sequences;
create and store data in a second index representing the second set of nucleic acid sequences;
identify a target region to serve as a nucleic acid reference sequence that corresponds to one or more of the nucleic acid sequences in the first set and that discriminates against one or more of the nucleic acid sequences in the second set, wherein the identifying comprises:
identifying, by the first index, the target region as a conserved region appearing in every nucleic acid sequence in the first set; and
confirming, by the second index, that the conserved region appears in none of the nucleic acid sequences in the second set; and
transmit data representing the target region to a portable kit for storage on memory of the portable kit, wherein the portable kit is configured to compare the target region to a sample nucleic acid sequence to determine whether the target region matches the sample nucleic acid sequence.


SEQUENCE LISTING (14 sequences)

SEQ ID NO: 1 - DNA, 16 bp, synthetic construct
  source 1..16: mol_type = other DNA
attgcttccatgggtc

SEQ ID NO: 2 - protein, 108 aa, synthetic construct
DVQMIQSPSSLSASLGDIVTMTCQASQGTSINLNWFQQKPGKAPKLLIYGSSNLEDGVPSRFSGSRYGTDFTLTISSLEDEDLATYFCLQHSYLPYTFGGGTKLEIKR

SEQ ID NO: 3 - DNA, 16 bp, synthetic construct
  source 1..16: mol_type = other DNA
aaaaaaaaaaaaaaaa

SEQ ID NO: 4 - DNA, 16 bp, synthetic construct
  source 1..16: mol_type = other DNA
aaaaaaaaaaaaaatt

SEQ ID NO: 5 - DNA, 16 bp, synthetic construct
  source 1..16: mol_type = other DNA
gtaagatccgctacaa

SEQ ID NO: 6 - DNA, 16 bp, synthetic construct
  source 1..16: mol_type = other DNA
cccccccccccccccc

SEQ ID NO: 7 - DNA, 23 bp, synthetic construct
  source 1..23: mol_type = other DNA
atccgctaatgcgccgtagtccg

SEQ ID NO: 8 - DNA, 16 bp, synthetic construct
  source 1..16: mol_type = other DNA
aaaaaaaaaaaaaaat

SEQ ID NO: 9 - DNA, 16 bp, synthetic construct
  source 1..16: mol_type = other DNA
aaaaaaaaaaaaaata

SEQ ID NO: 10 - DNA, 16 bp, synthetic construct
  source 1..16: mol_type = other DNA
gtaagatccgctacat

SEQ ID NO: 11 - DNA, 16 bp, synthetic construct
  source 1..16: mol_type = other DNA
gtaagatccgctacta

SEQ ID NO: 12 - DNA, 16 bp, synthetic construct
  source 1..16: mol_type = other DNA
cgcccccccccccccg

SEQ ID NO: 13 - DNA, 16 bp, synthetic construct
  source 1..16: mol_type = other DNA
cgccccccccccccgc

SEQ ID NO: 14 - DNA, 16 bp, synthetic construct
  source 1..16: mol_type = other DNA
cgcccccccccccccc